(19) United States
(12) Patent Application Publication
(10) Pub. No.: US 2023/0200435 A1
(43) Pub. Date: Jun. 29, 2023

(54) CARYOPHYLLENE-CONTAINING AGENT OR COMPOSITION AND VARIOUS APPLICATIONS THEREOF

(71) Applicants: KINKI UNIVERSITY, Osaka (JP); INABATA KORYO CO., LTD., Osaka (JP); SUNSHO PHARMACEUTICAL CO., LTD., Shizuoka (JP)

(72) Inventors: Nobuhiro ZAIMA, Nara (JP); Yuri YOSHIOKA, Osaka (JP); Shinichi MATSUMURA, Osaka (JP); Kohei IWAMOTO, Osaka (JP); Kazuya YAMADA, Shizuoka (JP); Takanori KOBAYASHI, Shizuoka (JP)

(73) Assignees: KINKI UNIVERSITY, Osaka (JP); INABATA KORYO CO., LTD., Osaka (JP); SUNSHO PHARMACEUTICAL CO., LTD., Shizuoka (JP)

(21) Appl. No.: 17/910,672

(22) PCT Filed: Mar. 10, 2021
(86) PCT No.: PCT/JP2021/009648

§ 371 Date: Sep. 9, 2022

Related U.S. Application Data
(60) Provisional application No. 62/987,548, filed on Mar. 10, 2020.

(51) Int. Cl. A24D 3/14; A24D 1/00; C11B 9/00; A23L 29/00; A61K 8/31; A61Q 19/00; A61Q 11/00; A61K 9/48; A61K 31/015

(52) U.S. Cl. CPC A24D 3/14; A23L 29/03; A24D 1/002; A61K 8/31; A61K 9/48; A61K 31/015; A61Q 11/00; A61Q 19/00; C11B 9/0007

(57) ABSTRACT

Provided are a novel agent or composition and others.

The agent or composition comprises caryophyllene and is used for at least one selected from the following (purposes) (1) to (3) :
(1) promoting relaxation, prolonging a resting-state time, and/or prolonging a motionless time,
(2) promoting sleep, and
(3) preventing blood pressure elevation.

The present invention also relates to a method for inhaling β-caryophyllene simultaneously with smoking a tobacco product, characterized in that the tobacco product contains a β-caryophyllene-containing seamless capsule in a tobacco filter, wherein the seamless capsule contains an essential oil containing β-caryophyllene as an active ingredient. The applicants found that pulmonary inhalation of β-caryophyllene by the above method allows β-caryophyllene to be efficiently distributed throughout the body, leading to achievements of relaxing and sleep-inducing effects, etc.

### TECHNICAL FIELD

The present invention relates to a technology or product for promoting sleep induction and relaxation using caryophyllene (such as β-caryophyllene) (e.g., a composition having relaxing and sleep-inducing effects, and a tobacco capsule, filter, food or drink product, or air freshener containing the composition).

### BACKGROUND ART

β-Caryophyllene has been known to prevent sleep disorders by relieving anxiety (Patent Literature 1). For example, an invention is disclosed relating to a poultry and livestock feed for relieving stress which comprises 0.0002 to 0.00375 mass% β-caryophyllene (Patent Literature 2).

In addition, β-caryophyllene reportedly binds to type 2 cannabinoid (CB2) receptor (Non-Patent Literature 1). Cannabinoids are a group of compounds found in cannabis plants and have sedative effects. β-Caryophyllene does not bind to type 1 cannabinoid (CB1) receptor expressed in the central nervous system and is considered to be non-addictive. Instead, β-caryophyllene binds to CB2 receptor and has inhibitory effects on inflammation and pain. β-Caryophyllene is present in natural essential oils (clove oil, copaiba oil, basil oil, oregano oil, hop oil, cinnamon oil, rosemary oil, black pepper oil, lavender oil) and is highly safe.

### CITATION LIST

### Patent Literature

Patent Literature 1: JP-A 2006-342062
Patent Literature 2: JP-A 2008-19251

### Non-Patent Literature

Non-Patent Literature 1:

Gertsch, J., et al., Proc. Natl. Ac. Sci., 2008, 105, 9099-9104.

### SUMMARY OF INVENTION

### Technical Problem

The application of caryophyllene (such as β-caryophyllene) for preventing sleep disorders and relieving stress in livestock, as mentioned above, has been increasingly studied.

However, such studies are still in progress, and specific effects of caryophyllene, such as relaxing and sleep-inducing effects, are unknown.

In addition, the characteristics of caryophyllene have not been sufficiently investigated in terms of its formulation, mode of delivery, pharmacokinetics, bioavailability, safety, and other details, partly because studies on caryophyllene are still in progress.

Under such circumstances, novel functions of caryophyllene, novel formulations (compositions) containing caryophyllene, and novel technologies relating to caryophyllene have been desired.

In response to this issue, an object of the present invention is to provide novel functions of caryophyllene (applications of caryophyllene based on their novel functions).

Another object of the present invention is to provide a novel composition (formulation) comprising caryophyllene.

### Solution to Problem

After intensive studies to achieve the above-mentioned objects, the present inventors found that caryophyllene (such as β-caryophyllene) has the following functions: relaxing effect (relaxation promoting effect, relaxation promoting function), sleep inducing effect [sleep promoting function, sleep improving function, sleep inducing function], blood pressure reducing effect [antihypertensive effect, preventive effect on blood pressure elevation, blood pressure reducing function], etc.

In addition, the present inventors found that a novel agent or formulation (composition) can be provided based on the form in which caryophyllene is contained; and that selecting such a formulation allows caryophyllene to be efficiently delivered and to efficiently achieve its functions (for example, in the case where β-caryophyllene is contained in the shell of a seamless capsule, which is then embedded in a filter for inhalation, β-caryophyllene can efficiently be delivered into the body by inhalation).

That is, the present invention relates to the following.
An agent or composition for promoting relaxation, prolonging a resting-state time, and/or prolonging a motionless time, comprising caryophyllene.
An agent or composition for promoting sleep (or for inducing sleep, for shortening sleep latency, or for prolonging sleep time), comprising caryophyllene.
An agent or composition for preventing blood pressure elevation, comprising caryophyllene.
An agent or composition comprising caryophyllene for at least one selected from oral, pulmonary (inhalation), and dermal delivery (for delivery via at least one selected from oral, pulmonary, and dermal routes).
A composition (a flavoring composition, a caryophyllene-containing flavoring composition) comprising caryophyllene and a flavoring agent.
An agent or composition comprising caryophyllene for an application selected from capsules, filters, tobacco products, inhalation devices, perfumery or cosmetic products, and food or drink products.
The agent or composition according to any one of the above [1] to [6], wherein the amount of the caryophyllene is 1 mass% or more relative to the total mass of the agent or composition which is assumed as 100 mass%.
A capsule containing caryophyllene.
A capsule having a core (content, liquid content) and a shell, wherein the core (content) contains caryophyllene.
A filter containing caryophyllene.
A filter containing a capsule (a capsule-embedded filter, a filter composed of a capsule-embedded filtration member), wherein the capsule at least comprises a first capsule, wherein the first capsule has a core (content, liquid content) and a shell, and wherein the core (content) contains caryophyllene.
The filter according to the above [11], wherein the capsule further comprises a second capsule, and wherein the second capsule has a content different from that of the first capsule.
The capsule or filter according to any one of the above [9], [11], and [12], wherein the core contains 1 mass% or more caryophyllene relative to the total mass of the core which is assumed as 100 mass%.
The capsule or filter according to any one of the above [9], and [11] to [13], wherein the core further contains a carrier and/or a flavoring agent.
The filter according to any one of the above [11] to [14], wherein the second capsule has a core (content, liquid content) and a shell, wherein the core (content) of the second capsule at least contains a flavoring agent.
The agent, composition, capsule, or filter according to any one of the above [1] to [15], wherein the caryophyllene includes a caryophyllene extracted or concentrated from clove, caraway, basil, oregano, hop, cinnamon, Ceylon cinnamon, rosemary, cannabis, hemp, Cannabis sativa, black pepper, lavender, malabathrum, ylang-ylang, copaiba, melegueta pepper, or other essential oils.
The agent, composition, capsule, or filter according to any one of the above [1] to [16], wherein the caryophyllene includes a chemically synthesized caryophyllene.
The agent, composition, capsule, or filter according to any one of the above [4] to [17], for at least one selected from the following (purposes) (1) to (3):
   (1) promoting relaxation, prolonging a resting-state time, and/or prolonging a motionless time,
   (2) promoting sleep, and
   (3) preventing blood pressure elevation.
The agent, composition, capsule, or filter according to any one of the above [1] to [18], wherein the agent, composition, capsule, or filter is for pulmonary (inhalation) delivery.
The agent, composition, capsule, or filter according to any one of the above [1] to [19], wherein the caryophyllene is pulmonarily delivered at a rate of 0.1 mg/min or more.
The agent, composition, or capsule according to any one of the above [1] to [18], wherein the caryophyllene is orally delivered in a dose of 1 mg or more.
A tobacco product containing caryophyllene.
An inhalation device containing caryophyllene.
The inhalation device according to the above [23], wherein the inhalation device is a smoking device (e.g., an e-cigarette or a heated tobacco product).
The tobacco product or inhalation device according to any one of the above [22] to [24], wherein the tobacco product or inhalation device contains the capsule or filter according to any one of the above [8] to [18].
A perfumery or cosmetic product containing caryophyllene.
The perfumery or cosmetic product according to the above [26], wherein the perfumery or cosmetic product is an air freshener.
The perfumery or cosmetic product according to the above [26], wherein the perfumery or cosmetic product is an oral care product.
The perfumery or cosmetic product according to the above [26], wherein the perfumery or cosmetic product is a cosmetic product.
A food or drink product containing caryophyllene.
The food or drink product according to the above [30], wherein the food or drink product is in the form of a capsule.
The tobacco product, inhalation device, perfumery or cosmetic product, or food or drink product according to any one of the above [22] to [31], for at least one selected from the following (purposes) (1) to (3):
   (1) promoting relaxation, prolonging a resting-state time, and/or prolonging a motionless time,
   (2) promoting sleep, and
   (3) preventing blood pressure elevation.
A method for promoting relaxation, prolonging a resting-state time, and/or prolonging a motionless time by delivery of caryophyllene (a caryophyllene-containing agent or composition).
A method for inducing sleep by delivery of caryophyllene (a caryophyllene-containing agent or composition).
A method for preventing blood pressure elevation by delivery of caryophyllene (a caryophyllene-containing agent or composition).
The method according to any one of the above [33] to [35], wherein the delivery is at least one selected from oral, pulmonary, and dermal delivery.
The method according to any one of the above [33] to [36], wherein the delivery is pulmonary (inhalation) delivery.
The method according to any one of the above [33] to [37], wherein the delivery is pulmonary delivery using a capsule or filter containing caryophyllene.
The method according to any one of the above [33] to [38], wherein the delivery is (at least) pulmonary delivery using (through) a tobacco product, an inhalation device, and/or a perfumery or cosmetic product each containing caryophyllene.
The method according to any one of the above [33] to [39], wherein the delivery is pulmonary delivery using the capsule or filter according to any one of the above [9], and [11] to [17], and wherein the delivery is achieved by breaking the capsule containing caryophyllene in the core (content) (and then inhaling).
The method according to any one of the above [33] to [36], wherein a food or drink containing caryophyllene is orally delivered.

### Claim 1

A composition for promoting relaxation, comprising β-caryophyllene as an active ingredient.

### Claim 2

A composition for inducing sleep, comprising β-caryophyllene as an active ingredient.

### Claim 3

The composition according to claim 1 or 2, wherein the amount of the β-caryophyllene is 20 to 100% of the amount of the composition which is assumed as 100 mass%.

### Claim 4

The composition according to claim 3, wherein the β-caryophyllene includes a β-caryophyllene extracted or concentrated from clove, caraway, basil, oregano, hop, cinnamon, Ceylon cinnamon, rosemary, cannabis, hemp, Cannabis sativa, black pepper, lavender, malabathrum, ylang-ylang, copaiba, melegueta pepper, or other essential oils.

### Claim 5

The composition according to claim 3, wherein the β-caryophyllene includes a chemically synthesized β-caryophyllene.

### Claim 6

A capsule having a shell and a liquid content entrapped in the shell, the liquid content containing the composition according to any one of claims 1 to 5, wherein the amount of the composition in the liquid content is 20 to 100% of the amount of the liquid content which is assumed as 100%.

### Claim 7

The capsule according to claim 6, wherein the amount of the composition in the liquid content entrapped in the shell is less than 100%, and wherein an additional composition in the liquid content entrapped in the shell is a solvent and/or a flavoring agent.

### Claim 8

A filter for an inhalation device, comprising a first capsule having a shell and a liquid content entrapped in the shell, the liquid content at least containing the composition of any one of claims 1 to 5; a second capsule having a shell and a liquid content entrapped in the shell, the liquid content being different from that of the first capsule; and a filtration member having the first and second capsules embedded therein.

### Claim 9

The filter for an inhalation device according to claim 8, wherein the second capsule at least contains a flavoring agent.

### Claim 10

A tobacco product having the filter for an inhalation device according to claim 8.

### Claim 11

An inhalation device having the filter for an inhalation device according to claim 8.

### Claim 12

A method for promoting relaxation by pulmonary delivery of β-caryophyllene by inhalation through a filter of an inhalation device.

### Claim 13

A method for inducing sleep by pulmonary delivery of β-caryophyllene by inhalation through a filter of an inhalation device.

### Advantageous Effects of Invention

The present invention provides novel applications (functions, agents) of caryophyllene, such as relaxation, sleep promotion (sleep induction), and blood pressure reduction.

In another aspect of the present invention, a novel agent or composition (formulation) comprising caryophyllene is provided. The novel agent or composition (formulation) can be used in various applications (e.g., capsule contents, tobacco products, inhalation devices, perfumery or cosmetic products, food or drink products, etc.).

In another aspect of the present invention, selecting the mode of use of caryophyllene allows caryophyllene to be efficiently delivered and to efficiently achieve its functions.

For example, caryophyllene can be used in the content (core) of capsules, which are to be broken in use, or caryophyllene can be used in inhalation devices (such as e-cigarettes and heated tobacco products) and air fresheners. In such modes of use, caryophyllene can efficiently be delivered via a pulmonary route.

In addition, caryophyllene can be used in oral agents (oral compositions) and cosmetic products, and thereby can be delivered by absorption through the mucosa (such as oral mucosa) and skin, as well as through a pulmonary route.

Furthermore, caryophyllene can be used in food or drink products and thereby can be orally delivered.

According to the present inventors’ study, pulmonary delivery of caryophyllene, among other delivery (administration) routes, leads to efficient achievement of its functions (e.g., relaxation, sleep promotion (sleep induction), blood pressure reduction, etc.).

Therefore, according to the present invention, selecting such an advantageous pulmonary delivery allows caryophyllene to efficiently achieve its functions (e.g., a technology for delivering β-caryophyllene with high bioavailability can be provided).

### BRIEF DESCRIPTION OF DRAWINGS

FIG. 1 is a graph showing the device used in Experiment A (and a photograph of the device) and the spatial concentration of caryophyllene measured in Experiment A.

FIG. 2 is a graph showing the concentrations of caryophyllene in the serum, liver, and brain after different durations of caryophyllene exposure (inhalation) in Experiment 1.

FIG. 3 is a graph showing the time-course change in the concentrations of caryophyllene in the serum, liver, and brain after 60 minutes of caryophyllene exposure (inhalation) in Experiment 2.

FIG. 4 is a graph showing the motionless time and the sleep time in the 60-min caryophyllene exposure (inhalation) group and the control group (non-exposure group) during 3, 600-second monitoring in Experiment 3.

FIG. 5 illustrates the results obtained in Example 2.

FIG. 6 is a graph showing the estimated serum concentration of β-caryophyllene in smoking one cigarette per hour, 20 cigarettes in total per day in Experiment 11.

### DESCRIPTION OF EMBODIMENTS

Hereinafter, the present invention will be described in detail.

The agent or composition of the present invention (including its specific applications (applied products) such as a capsule, a filter, an inhalation device, a perfumery or cosmetic product, or a food or drink product; hereinafter the same applies to the description for the “agent or composition”) comprises caryophyllene.

### Caryophyllene

Examples of the caryophyllene include β-caryophyllene, α-caryophyllene, isocaryophyllene, and metabolites or derivatives of caryophyllene (e.g., caryophyllene oxides such as β-caryophyllene oxide). The caryophyllene may comprise a single type of caryophyllene or a combination of two or more types of caryophyllenes selected from the above examples.

The caryophyllene usually may at least comprise β-caryophyllene. The caryophyllene may comprise β-caryophyllene and a type of caryophyllene that is not β-caryophyllene [e.g., at least one selected from α-caryophyllene, isocaryophyllene, and metabolite or derivatives of caryophyllene].

The amount of β-caryophyllene in the caryophyllene at least comprising β-caryophyllene is, for example, 30 mass% or more, 50 mass% or more, 70 mass% or more, 80 mass% or more, 90 mass% or more, 95 mass% or more, 100 mass% (substantially 100 mass%), etc.

As used herein, the term “β-caryophyllene” is sometimes used to collectively refer to all caryophyllenes, including those that are not β-caryophyllene.

The caryophyllene (β-caryophyllene) is not particularly limited and may be derived (e.g., extracted or concentrated) from, for example, clove, caraway, basil, oregano, hop, cinnamon, Ceylon cinnamon, rosemary, cannabis, hemp, Cannabis sativa, black pepper, lavender, malabathrum, ylang-ylang, copaiba, melegueta pepper, or other essential oils.

The caryophyllene may be a commercial product or may be produced (purified) by commonly used methods (chemically synthesized).

### Functions

The agent or composition of the present invention can be used for the purpose of providing (or achieving) various functions (effects).

The functions (use) include functions to reduce (ameliorate or suppress) anxiety [e.g., motion sickness, nocturia, stress urticaria, and sleep disorders], reduce (ameliorate or suppress) stress, inhibit β-secretase (β-secretase activity), and ameliorate major neurocognitive disorder (or dementia, for example, senile dementia such as Alzheimer’s disease). The agent or composition may be used, particularly, for at least one selected from the following purposes (functions or use) (1) to (3):
(1) promoting relaxation,
(2) promoting sleep (inducing sleep), and
(3) preventing blood pressure elevation.

The relaxation may be confirmed, for example, using a resting-state time (motionless time) as an indicator.

Therefore, the “promoting relaxation” can also be referred to as increasing (prolonging or extending) a resting-state time (motionless time).

In addition, the relaxation may be confirmed (directly or indirectly), for example, using other indicators such as an increase in skin temperature (facial skin temperature) (e.g., see references A and B below), an increase in body temperature (e.g., see reference C below), a decrease in heart rate, and/or an EEG pattern (e.g., α waves are dominant compared to β waves, see reference D below).

Among these indicators, a decrease in heart rate was reported to be associated with relaxation through grooming behaviors (e.g., see reference E below), as well as an increase in body temperature was reported in relation to behaviors observed during relaxation (e.g., see reference F below).

Thus, the relaxation can be confirmed (recognized) based on behavioral observation, such as measurement of a resting-state time (motionless time) as described in the EXAMPLES section below.

### Reference A

Hiroki Ito, Shizuka Bando, Kosuke Oiwa, Akio Nozawa: “Evaluation of Variations in Autonomic Nervous System’s Activity During the Day Based on Facial Thermal Images Using Independent Component Analysis”, IEEJ Transactions on Electronics, Information and Systems, Vol. 138, No. 7, pp.812-821 (2018)

### Reference B

Hiroko Adachi, Kosuke Oiwa, Akio Nozawa: “Study of reproducibility of sleepiness level determination model based on facial thermal images using CNN”, the 2017 IEEJ Electronics, Information and Systems Society Conference, TC16-3 (2017)

### Reference C

Takako Shimada: “Relationships between the affects of comfort and discomfort and deep skin temperature and the skin conductance level”, Japanese Journal of Nursing Art and Science, Vol. 3, No. 2, pp5-12, 2004

### Reference D

Kimihiko Ohtsuka, Shozo Kudo, Tohio Takiguchi, Yutaka Ohkuma: “The Relaxing Effect of Gum Chewing”, Journal of Japanese Society for Mastication Science and Health Promotion, Vol. 7, No. 1, pp11-16, 1997

### Reference E

Aureli, F.; Preston, S. D.; de Waal, F. B.: “Heart rate responses tosocial interactions in free-moving rhesus macaques (Macaca mulatta): a pilot study”, Journal of Comparative Psychology, Vol. 113, pp59-65, 1999

### Reference F

Yutaro Sato, Fumihiro Kano, Satoshi Hirata: “Cutting-edge infrared thermography as a new tool to explore animal emotions”, Japanese Journal of Animal Psychology, Vol. 68, No .1, pp 1-15, 2018

The sleep promotion may be confirmed, for example, using a sleep latency (time to sleep onset) and/or a sleep time as an indicator.

Therefore, the “promoting sleep” can also be referred to as shortening (reducing) a sleep latency (time to sleep onset) or increasing (prolonging or extending) a sleep time.

### Additional Ingredients, Forms, Applications, etc.

The agent or composition of the present invention is not particularly limited as long as it comprises caryophyllene. The agent or composition may be an agent (e.g., a liquid formulation) or composition simply comprising caryophyllene or may be in a form (composition) comprising caryophyllene together with additional ingredients.

The additional ingredient is not particularly limited and can be selected according to the desired function, form, application, applied product of the agent or composition, and other factors. Examples include carriers, excipients, binders, disintegrants, lubricants, coating agents, colorants, flavoring agents, stabilizers, emulsifiers, surfactants, absorption enhancers, gelling agents, pH adjusters, preservatives, antioxidants, coolants, physiologically active substances, biologically active substances, microorganisms, foods and drinks, plants, sweeteners, acidulants, seasonings, and revitalizers. A single kind of additional ingredient or a combination of two or more kinds of additional ingredients may be used.

Examples of the carrier (medium) include acids (e.g., fatty acids such as caprylic acid, capric acid, eicosapentaenoic acid, docosahexaenoic acid, oleic acid, and linoleic acid), esters {e.g., fats or oils [e.g., vegetable oils (e.g., soybean oil, rapeseed oil, corn oil, sesame oil, linseed oil, cotton seed oil, perilla oil, olive oil, rice oil, palm oil, jojoba oil, sunflower oil, camellia oil, etc.), animal oils (e.g., beef fat, pork fat, chicken fat, milk fat, fish oil, horse oil, etc.), and medium-chain triglycerides (MCTs)], non-glycerol esters (e.g., fatty acid esters such as octyldodecyl myristate and isopropyl myristate), etc.}, hydrocarbons (e.g., liquid paraffin, squalane, and vaseline), higher alcohols (e.g., cetostearyl alcohol, behenyl alcohol, etc.), silicones (e.g., silicone oil, etc.), synthetic polymers (e.g., polyacrylic acid, carboxyvinyl polymer, polyethylene glycol, polyvinylpyrrolidone, etc.), natural polymers or their derivatives (e.g., carrageenan, alginic acid, cellulose, guar gum, xanthan gum, quince seed, dextran, gellan gum, hyaluronic acid, ethyl cellulose, hydroxypropyl cellulose, hydroxypropyl methyl cellulose, cationic guar gum, acetylated hyaluronic acid, sodium alginate, etc.), lower alcohols (e.g., ethanol, isopropanol, etc.), polyhydric alcohols (e.g., glycerol, propylene glycol, butylene glycol, diglycerol, and dipropylene glycol), ethers (e.g., glycol ethers such as ethylene glycol monomethyl ether, ethylene glycol monoethyl ether, diethylene glycol monomethyl ether, diethylene glycol monopropyl ether, diethylene glycol monobutyl ether, propylene glycol monoethyl ether, and dipropylene glycol monoethyl ether), sugars and sugar alcohols (e.g., glucose, sucrose, sorbitol, dextrin, maltodextrin, etc.), and water.

The properties of the carrier can be selected according to the dosage form, the mode of delivery, and other factors. The carrier may be in a solid, liquid, or some other form, and may be non-volatile or volatile. A liquid carrier can be a solvent.

The flavoring agent (a flavoring agent that is not caryophyllene) may be a synthetic or natural flavoring agent, a blended flavor, a flavoring composition, etc.

The flavoring agent is not particularly limited as long as it can be used as an ingredient having an aroma, a flavor, etc.

Examples of the synthetic flavoring agent (or a component of the natural flavoring agent) include esters, alcohols, aldehydes, ketones, phenols, ethers, lactones, hydrocarbons, nitrogen-containing and/or sulfur-containing compounds, and acids.

Examples of the ester (e.g., a fatty acid or aromatic carboxylic acid ester) include, but are not particularly limited to, propyl formate, terpinyl formate, ethyl acetate, octyl acetate, nonyl acetate, decyl acetate, dodecyl acetate, dihydromyrcenyl acetate, linalyl acetate, citronellyl acetate, geranyl acetate, neryl acetate, tetrahydromugol acetate, lavandulyl acetate, nerolidyl acetate, dihydrocuminyl acetate, terpinyl acetate, citryl acetate, nopyl acetate, dihydroterpinyl acetate, 2,4-dimethyl-3-cyclohexenyl methyl acetate, myraldyl acetate, veticol acetate, decenyl propionate, linalyl propionate, octyl butyrate, cinnamyl butyrate, isopropyl isobutyrate, octyl isobutyrate, linalyl isobutyrate, 2-methylpentyl 2-methylvalerate, methyl 3-hydroxyhexanoate, methyl octanoate, methyl nonanoate, methyl undecylenate, linalyl benzoate, methyl cinnamate, isoprenyl angelate, methyl geranate, triethyl citrate, ethyl acetoacetate, ethyl 2-hexylacetoacetate, ethyl benzylacetoacetate, allyl 2-ethylbutyrate, ethyl 3-hydroxybutyrate, ethyl nonanoate, ethyl decanoate, ethyl 2,4-decadienoate, methyl anthranilate, and ethyl N-methyl anthranilate.

Examples of the alcohol include, but are not particularly limited to, 3-heptanol, 3-octanol, 1-nonanol, 1-decanol, 1-undecanol, 1-dodecanol, prenol, 10-undecen-1-ol, dihydrolinalool, tetrahydromugol, myrcenol, dihydromyrcenol, tetrahydromyrcenol, ocimenol, terpineol, 3-thujanol, benzyl alcohol, β-phenylethyl alcohol, trans-2-hexenol, cis-4-hexenol, citronellol, rhodinol, geraniol, nerol, linalool, tetrahydrolinalool, dimethyloctanol, hydroxycitronellol, isopulegol, menthol, terpineol, dihydroterpineol, carveol, dihydrocarveol, perilla alcohol, 4-thujanol, myrtenol, α-fenchyl alcohol, farnesol, nerolidol, cedrenol, anise alcohol, hydratropic alcohol, 3-phenylpropyl alcohol, cinnamyl alcohol, and amylcinnamyl alcohol.

Examples of the aldehyde include, but are not particularly limited to, acetaldehyde, n-hexanal, n-heptanal, n-octanal, n-nonanal, decanal, undecanal, tridecanal, tetradecanal, trans-2-hexenal, cis-4-decenal, 10-undecenal, trans-2-dodecenal, 3-dodecenal, trans-2-tridecenal, 2,4-hexadienal, 5,9-dimethyl-4,8-decadienal, citral, α-methylenecitronellal, citronellyl oxyacetaldehyde, myrtenal, neral, α- or β-sinensal, myrac aldehyde, phenylacetaldehyde, octanal dimethyl acetal, n-valeraldehyde, isovaleraldehyde, 2-methylbutanal, citronellal, hydroxycitronellal, safranal, vernaldehyde, benzaldehyde, phenylpropionaldehyde, cinnamaldehyde, salicylaldehyde, anisaldehyde, p-methylphenoxyacetaldehyde, acetaldehyde diethyl acetal, 2-phenyl-2,4-pentanediol acetal, 2-hexenal diethyl acetal, and 2-hexyl-5-methyl-1,3-dioxolane.

Examples of the ketone include, but are not particularly limited to, 2-pentanone, 3-heptanone, 3-octanone, 2-nonanone, 2-undecanone, 2-tridecanone, methylheptenone, dimethyloctenone, geranylacetone, 2,3,5-trimethyl-4-cyclohexenyl-1-methylketone, nerone, nootkatone, dihydronootkatone, acetophenone, 4,7-dihydro-2-isopentyl-2-methyl-1,3-dioxepin, 2,3-hexadione, ethyl isoamyl ketone, diacetyl, amylcyclopentenone, 2-cyclopentylcyclopentanone, hexylcyclopentanone, heptylcyclopentanone, cis-jasmone, dihydrojasmone, trimethylpentylcyclopentanone, α-dynascone, trimethylcyclohexenylbutenone, ionone, allyl ionone, plicatone, cashmeran, 1-carvone, menthone, camphor, p-methylacetophenone, p-methoxyacetophenone, benzylideneacetone, raspberry ketone, methylnaphthyl ketone, benzophenone, furfural acetone, homofuronol, maltol, ethylmaltol, and ethyl acetoacetate ethyleneglycol ketal.

Examples of the phenol include, but are not particularly limited to, thymol, carvacrol, β-naphthol isobutyl ether, anethole, β-naphthol methyl ether, β-naphthol ethyl ether, guaiacol, creosol, veratrole, hydroquinone dimethyl ether, 2,6-dimethoxyphenol, 4-ethylguaiacol, eugenol, isoeugenol, ethyl isoeugenol, and tert-butyl hydroquinone dimethyl ether.

Examples of the ether include, but are not particularly limited to, decyl vinyl ether, α-terpinyl methyl ether, isoproxen, 2,2-dimethyl-5-(1-methyl-1-propenyl)-tetrahydrofuran, rosefuran, 1,4-cineole, nerol oxide, 2,2,6-trimethyl-6-vinyltetrahydropyran, methylhexyl ether, ocimene epoxide, limonene oxide, Rhubofix (registered trademark), caryophyllene oxide, linalool oxide, 5-isopropenyl-2-methyl-2-vinyltetrahydrofuran, theaspirane, and rose oxide.

Examples of the lactone include, but are not particularly limited to, γ-undecalactone, δ-dodecalactone, γ-hexalactone, γ-nonalactone, γ-decalactone, γ-dodecalactone, jasmine lactone, methyl γ-decalactone, jasmolactone, propylidene phthalide, δ-hexalactone, δ-2-decenolactone, ε-dodecalactone, dihydrocoumarin, and coumarin.

Examples of the hydrocarbon include, but are not particularly limited to, ocimene, limonene, α-phellandrene, terpinene, 3-carene, bisabolene, valencene, alloocimene, myrcene, farnesene, α-pinene, β-pinene, camphene, terpinolene, p-cymene, cedrene, β-caryophyllene, and cadinene.

Examples of the nitrogen-containing and/or sulfur-containing compound include, but are not particularly limited to, methyl anthranilate, ethyl anthranilate, methyl N-methyl anthranilate, methyl N-2′-methylpentylideneanthranilate, ligantraal (trade name), dodecanitrile, 2-tridecenitrile, geranylnitrile, citronellylnitrile, 3,7-dimethyl-2,6-nonadienonitrile, indole, 5-methyl-3-heptanone oxime, limonenethiol, 1-P-menthene-8-thiol, butyl anthranilate, cis-3-hexenyl anthranilate, phenylethyl anthranilate, cinnamyl anthranilate, dimethyl sulfide, and 8-mercaptomenthone.

Examples of the acid include, but are not particularly limited to, acetic acid, propionic acid, butyric acid, valeric acid, hexanoic acid, octanoic acid, decanoic acid, dodecanoic acid, 2-decenoic acid, geranic acid, 2-methylbutyric acid, 2-ethylbutyric acid, phenylacetic acid, cinnamic acid, isobutyric acid, isovaleric acid, 3-methylvaleric acid, 2-hexenoic acid, 2-methyl-2-pentenoic acid, 2-methylheptanoic acid, myristic acid, stearic acid, lactic acid, pyruvic acid, and cyclohexanecarboxylic acid.

The natural flavoring agent (the original source of the natural flavoring agent) can be, for example, from mint, herbs, citrus, and many others, and is not particularly limited.

Examples of the natural flavoring agent (the original source of the natural flavoring agent) include sweet orange, bitter orange, neroli, mandarin, petitgrain, bergamot, tangerine, satsuma orange, daidai, hassak, iyokan, lemon, lime, grapefruit, yuzu, sudachi, kabosu, sweetie, citronella, elemi, olibanum, marjoram, angelica root, star anise, basil, hay, calamus, caraway, cardamom, pepper, cascarilla, ginger, sage, clary sage, clove, coriander, eucalyptus, fennel, pimento, juniper, fenugreek, laurel, mace, Japanese cedar, senkyu, almond, apple mint, anise, artemisia, alfalfa, apricot, ambrette, rush, strawberry, fig, ylang-ylang, wintergreen, ume, elder, enju, oakmoss, allspice, orris, currant, cassie, chamomile, galanga, Chinese quince, gambir, guava, gooseberry, camphor tree, gardenia, cubeb, cumin, cranberry, cola, Japanese pepper, sandarac, sandalwood, red sandalwood, perilla, civet, jasmine, ginger, ginseng, cinnamon, starfruit, styrax, spearmint, geranium, thyme, davana, tansy, champaca, tuberose, camellia, dittany, tolu balsam, tonka beans, nut, jujube, nutmeg, nanten, niaouli, carrot, violet, pineapple, hibiscus, honey, Japanese mint, passion fruit, vanilla, rose, hyssop, hinoki cypress, fusel oil, buchu, peppermint, pepino, verbena, rosewood, pawpaw, boldo, boronia, pine, mango, beeswax, mimosa, milfoil (yarrow), musk, maple, melissa (lemon balm), melon, peach, lavender, liqueur, litsea, linden, rue, wax jambu, rosemary, and lovage.

Specific examples of the flavoring agent (flavoring composition) include citrus flavors such as orange flavor, lemon flavor, lime flavor, grapefruit flavor, yuzu flavor, and sudachi flavor; berry flavors such as strawberry flavor, raspberry flavor, and blueberry flavor; tropical fruit flavors such as mango flavor, papaya flavor, guava flavor, passion fruit flavor, and lychee flavor; fruit flavors such as apple flavor, grape flavor, pineapple flavor, banana flavor, peach flavor, melon flavor, apricot flavor, plum flavor, and cherry flavor; tea and coffee flavors such as green tea flavor, oolong tea flavor, black tea flavor, and coffee flavor; meat flavors such as beef flavor, pork flavor, and chicken flavor; herbal and spice flavors such as asafetida flavor, ajowan flavor, anise flavor, angelica flavor, fennel flavor, allspice flavor, cinnamon flavor, cassia flavor, chamomile flavor, leaf mustard flavor, cardamom flavor, caraway flavor, cumin flavor, clove flavor, pepper flavor, coriander flavor, sassafras flavor, savory flavor, Japanese pepper flavor, perilla flavor, juniper berry flavor, ginger flavor, star anise flavor, horseradish flavor, sage flavor, thyme flavor, tarragon flavor, dill flavor, capsicum flavor, jujube flavor, nutmeg flavor, basil flavor, parsley flavor, marjoram flavor, rosemary flavor, laurel flavor, and wasabi flavor; vegetable flavors such as onion flavor, garlic flavor, leek flavor, cabbage flavor, carrot flavor, celery flavor, shiitake mushroom flavor, matsutake mushroom flavor, tomato flavor, burdock flavor, and Japanese honewort flavor; mint flavors such as peppermint flavor, spearmint flavor, and Japanese mint flavor; vanilla flavors; nut flavors such as almond flavor, cashew nut flavor, peanut flavor, hazelnut flavor, walnut flavor, chestnut flavor, macadamia nut flavor, pecan nut flavor, pistachio flavor, Brazil nut flavor, and coconut flavor; western liquor flavors such as wine flavor, whiskey flavor, brandy flavor, rum flavor, gin flavor, and liqueur flavor; seafood flavors such as fish flavor, shellfish flavor, dried fish flavor, and seaweed flavor; grain flavors such as corn flavor, potato flavor, sweet potato flavor, rice flavor, and bread flavor; and sugar-based flavors such as honey flavor, maple syrup flavor, sugar flavor, brown sugar flavor, and molasses flavor.

The properties of the flavoring agent can be selected according to the dosage form, the mode of delivery, and other factors. The flavoring agent may be in a solid, liquid, or some other form, and may be non-volatile or volatile.

The agent or composition may be formulated into an appropriate dosage form according to the desired function, the mode of delivery, and other factors. The form (dosage form, properties) of the agent or composition (formulation) is not particularly limited, and examples include tablets, powders, fine granules, granules, dry syrups, coated tablets, orally disintegrating tablets, chewable tablets, capsules, soft capsules, syrups, oral liquids, lozenges, jellies, inhalations, suppositories, injections, ointments, eye drops, eye ointments, nasal drops, ear drops, patches, lotions, topical liquids, sprays, topical aerosols, creams, gels, tapes, buccal tablets, sublingual tablets, liquids, suspensions, emulsions, liniments, and sheets.

The mode of delivery (administration or intake) of the agent or composition into the body is not particularly limited, and oral delivery (administration) or parenteral delivery (administration) may be selected. For parenteral delivery (administration), for example, pulmonary administration, nasal administration, dermal administration, mucosal (e.g., oral mucosal) administration, ocular instillation, auricular instillation, or injection (subcutaneous injection, intramuscular injection, intravenous injection, etc.) can be used. A single one of these modes of delivery or a combination of two or more of them may be used.

Typical modes of delivery include oral, pulmonary, or dermal delivery. Particularly preferred is pulmonary delivery. Pulmonary delivery (e.g., inhalation) enables efficient delivery of caryophyllene. For this reason, the mode of delivery may be at least pulmonary delivery.

The mode of delivery may be selected as appropriate according to the application and purpose of the agent or composition to be delivered (the desired function of caryophyllene). For example, for at least one selected from the above-mentioned purposes (functions, applications) (1) to (3), pulmonary delivery is suitable because it seemingly allows the caryophyllene to efficiently (advantageously) exert (achieve) its functions.

The amount of the caryophyllene in the agent or composition of the present invention is not particularly limited and can be selected according to the dosage form, the mode of delivery, the amount delivered (administered), and other factors. For example, the amount of the caryophyllene may be 0.01 mass% or more (e.g., 0.05 mass% or more), 0.1 mass% or more (e.g., 0.5 mass% or more), 1 mass% or more (e.g., 5 mass% or more), 10 mass% or more (e.g., 15 mass% or more), 20 mass% or more (e.g., 25 mass% or more), etc. when the total mass of the agent or composition is assumed as 100 mass%.

In the case where the agent or composition comprises a carrier, the amount of the carrier is not particularly limited and can be selected according to the dosage form, the mode of delivery, the amount delivered (administered), and other factors. For example, the amount of the carrier relative to 1 part by mass of the caryophyllene may be 0.1 part by mass or more, 0.3 part by mass or more, 0.5 part by mass or more, 0.7 part by mass or more, 1 part by mass or more, 1.2 parts by mass or more, 1.5 parts by mass or more, 2 parts by mass or more, 3 parts by mass or more, etc. ; and may be 200 parts by mass or less, 150 parts by mass or less, 120 parts by mass or less, 100 parts by mass or less, 80 parts by mass or less, 50 parts by mass or less, 30 parts by mass or less, 20 parts by mass or less, 15 parts by mass or less, 10 parts by mass or less, etc.

In the case where the agent or composition comprises a flavoring agent (is a flavoring composition (such as a flavored liquid solution)), the amount of the flavoring agent is not particularly limited and can be selected according to the dosage form, the mode of delivery, the amount delivered (administered), and other factors. For example, the amount of the flavoring agent relative to 1 part by mass of the caryophyllene may be 0.01 part by mass or more, 0.05 part by mass or more, 0.1 part by mass or more, 0.5 part by mass or more, 1 part by mass or more, 1.2 parts by mass or more, 1.5 parts by mass or more, 2 parts by mass or more, 2.5 parts by mass or more, etc.; and may be 100 parts by mass or less, 80 parts by mass or less, 50 parts by mass or less, 30 parts by mass or less, 20 parts by mass or less, 15 parts by mass or less, 10 parts by mass or less, 8 parts by mass or less, 5 parts by mass or less, 3 parts by mass or less, 2 parts by mass or less, 1.5 parts by mass or less, etc.

The amount of the agent or composition delivered (administered, taken) can be selected according to the desired application/function and the dosage form (and the age, gender, and body weight of the subject, and other factors) and is not particularly limited.

For example, the agent or composition may be delivered in a dose of 0.01 mg or more, 0.05 mg or more, 0.1 mg or more, etc., in terms of caryophyllene.

In a specific mode, the agent or composition may be pulmonarily delivered (inhaled) at a rate of 0.1 mg/min or more in terms of caryophyllene.

The amount (volume) of the agent or composition (caryophyllene-containing vapor or gas) per inhalation may be, for example, 10 mL or more, 20 mL or more, 30 mL or more, etc., and may be 4,500 mL or less, 4,000 mL or less, 3,000 mL or less, 2,000 mL or less, 1,000 mL or less, 500 mL or less, etc.

In another specific mode, the agent or composition may be orally delivered in a dose of, for example, 1 mg or more in terms of caryophyllene.

The frequency of delivery (e.g., the frequency of delivery per day) of the agent or composition (caryophyllene) can be selected according to the mode of delivery, the desired function, and other factors, and may be once daily or several times daily.

The subject to which the agent or composition is to be delivered may be, for example, a human or a non-human animal. The non-human animal may be a pet animal (e.g., a dog, a cat, etc.).

The agent or composition (or caryophyllene) may be formulated into an appropriate dosage form as described above and can be used (applied) in various applications (products). Specific examples of the mode of use (application) include capsules (e.g., capsule contents), filters, tobacco products, inhalation devices, perfumery or cosmetic products, and food or drink products. In such applications (modes of use), the mode of delivery as described above (e.g., pulmonary delivery, oral delivery, etc.) or specific purposes (e.g., promoting relaxation, inducing sleep, and/or preventing blood pressure elevation) can be selected according to the type of application and other factors.

These examples will be described below.

### Capsules

The capsule may have a shell only or have a shell and a content (core). In particular, the capsule for tobacco products, etc., may have a core (content, inner liquid, inner substance) and a shell (outer layer, coat, capsule coat).

The capsule may be a soft capsule, a hard capsule, a seamless capsule, etc. In particular, the capsule for tobacco products, etc., may be a seamless capsule.

The caryophyllene may be contained in a capsule in any manner, whether in a shell, a core, or both. In particular, when the capsule is a capsule having a core (seamless capsule), the caryophyllene may be contained in the core (or at least in the core). In other words, the agent or composition of the present invention (caryophyllene) is used for a capsule content.

The shell (outer layer) usually may contain a shell-forming component (shell-forming base, shell-forming material). The shell-forming component is not particularly limited and can be selected as appropriate according to the application of the capsule and other factors. Examples of the shell-forming component include polysaccharides (or their derivatives) {e.g., polysaccharides from seaweeds [e.g., agar, carrageenan, alginic acid or its salts (e.g., alkali metal salts (a sodium salt, a potassium salt, etc.), alkaline earth metal salts (a calcium salt, a magnesium salt, etc.), an iron salt, a tin salt, and other metal salts), furcellaran, curdlan, etc.], polysaccharides from resins (e.g., gum ghatti, gum arabic, etc.), polysaccharides from microorganisms (e.g., pullulan, welan gum, xanthan gum, gellan gum, etc.), polysaccharides from plants (e.g., tragacanth gum, pectin, glucomannan, starch, polydextrose, dextrin, maltodextrin, cyclodextrin, indigestible dextrin, etc.), polysaccharides from seeds [e.g., guar gum or its derivatives (e.g., hydroxypropyl guar gum, cationic guar gum, and hydrolyzed guar gum (such as enzymatically hydrolyzed guar gum)), tara gum, tamarind seed gum, locust bean gum, psyllium seed gum, and linseed gum], fermented polysaccharides (e.g., diutan gum, etc.), cellulose derivatives (e.g., hydroxypropyl cellulose, hydroxypropylmethyl cellulose, methyl cellulose, carboxymethyl cellulose, etc.), chitosan, etc.}, synthetic resins (polyvinyl alcohol, etc.), proteins (e.g., gelatin, casein, zein, etc.), and sugar alcohols (e.g., sorbitol, maltitol, lactitol, hydrogenated isomaltulose, xylitol, mannitol, galactitol, erythritol, etc.).

A single kind of shell-forming component or a combination of two or more kinds of shell-forming components may be used.

The shell-forming component may be capable of forming hydrophilic colloids. Some types of shell-forming components can function, for example, as a plasticizer, a sweetener, a dietary fiber, or a bulking agent. The shell-forming component may be a commercial product.

The shell may contain a plasticizer, a colorant, a sweetener, a flavoring agent, an antioxidant, a preservative, and other components.

For example, the shell may contain a plasticizer for purposes including adjustment of shell strength. Specific examples of the plasticizer include polyhydric alcohols (e.g., (poly)alkylene glycols such as ethylene glycol, propylene glycol, polyethylene glycol, and polypropylene glycol; and polyols having three hydroxy groups or more, such as glycerol), sugars [e.g., monosaccharides (e.g., glucose, fructose, glucose, galactose, etc.), disaccharides (e.g., sucrose, maltose, trehalose, coupling sugar, etc.), oligosaccharides (e.g., maltooligosaccharides, etc.), etc.], sugar alcohols (e.g., sugar alcohols as exemplified above, such as sorbitol, maltitol, lactitol, hydrogenated isomaltulose, xylitol, mannitol, galactitol, and erythritol), polysaccharides or their derivatives [e.g., starch, starch derivatives (e.g., polydextrose, dextrin, maltodextrin, indigestible dextrin, cyclodextrins (α, β, and γ), etc.), cellulose derivatives (e.g., hydroxypropyl cellulose, hydroxypropylmethyl cellulose, methyl cellulose, carboxymethyl cellulose, etc.), etc.], polyvinyl alcohol, and triacetin. A single kind of plasticizer or a combination of two or more kinds of plasticizers may be used.

Sugar alcohols, starch, starch derivatives, etc., can be used also as the shell-forming component as described above.

In the capsule having a core, the core may be in a solid, liquid, or some other form. Particularly in the capsule for pulmonary caryophyllene delivery, the core may be in a liquid form. The liquid form includes a colloidal form, an emulsified form, and a gelatinous form.

The core may contain caryophyllene as described above, and may contain an additional ingredient.

The additional ingredient is, for example, a carrier [e.g., an acid or an ester, particularly a liquid carrier (e.g., a liquid fat or oil such as an MCT, or a liquid fatty acid)], a flavoring agent (e.g., menthol), or some other ingredient as exemplified above.

For example, a flavoring agent and caryophyllene may be both contained in a single capsule (capsule content) so that users can enjoy both the flavor of the flavoring agent and the effect of the caryophyllene.

Such a capsule containing a flavoring agent (e.g., a seamless capsule having a core containing a flavoring agent) can be also referred to as a flavored capsule.

The core usually may be non-soluble in (non-erosive to) the shell (or to the area in contact with the shell).

The amount of the caryophyllene in the capsule may be selected from the same ranges as described above. For example, the amount of the caryophyllene can be selected from a range of about 0.1 mass% or more (e.g., 0.5 mass% or more) relative to the total mass of the capsule, and is preferably 1 mass% or more (e.g., 2 mass% or more), more preferably 3 mass% or more (e.g., 5 mass% or more). The amount of the caryophyllene may be 10 mass% or more (e.g., 15 mass% or more, 20 mass% or more, 30 mass% or more, 50 mass% or more, etc.).

In the case where the core contains caryophyllene, the amount of the caryophyllene may be selected from the same ranges as described above. For example, the amount of the caryophyllene can be selected from a range of about 0.1 mass% or more (e.g., 0.5 mass% or more) relative to the total mass of the core (content), and is preferably 1 mass% or more (e.g., 2 mass% or more), more preferably 3 mass% or more (e.g., 5 mass% or more). The amount of the caryophyllene may be 10 mass% or more (e.g., 15 mass% or more, 20 mass% or more, 30 mass% or more, 50 mass% or more, etc.).

In the case where the core contains caryophyllene, the upper limit of the amount of the caryophyllene is not particularly specified and may be substantially 100 mass% (i.e., the core is made only of caryophyllene) relative to the total mass of the core (content) or may be less than 100 mass% (e.g., 95 mass% or less, 90 mass% or less, 80 mass% or less, etc.).

In the case where the core contains an additional ingredient, the amount of the additional ingredient is not particularly limited. For example, in the case where the core contains a carrier and/or a flavoring agent, the amount of the carrier and/or the flavoring agent relative to the total mass of the caryophyllene may be selected from the same ranges as described above.

The diameter (average diameter) of the capsule (or shell) can be selected as appropriate according to the type of capsule, the application, the mode of delivery of caryophyllene, and other factors. The diameter may be, for example, 0.1 mm or more, 0.5 mm or more, 1 mm or more, 1.5 mm or more, 2 mm or more, etc. In addition, the diameter may be 30 mm or less, 25 mm or less, 20 mm or less, 18 mm or less, 15 mm or less, 12 mm or less, 10 mm or less, 8 mm or less, etc. More specifically, the diameter may be 2.8 mm, 3.0 mm, 3.4 mm, 3.5 mm, 4.0 mm, etc., but is not limited thereto.

The percentage of the shell in the capsule having a core (the proportion of the shell relative to the whole of the capsule (the combined amount of the shell and the capsule content)) may be selected from, for example, a range of about 0.1 to 99 mass% (e.g., 0.5 to 95 mass%). The shell percentage is, for example, about 1 to 90 mass%, preferably about 1.5 to 80 mass% (e.g., 2 to 70 mass%), and more preferably about 2.5 to 60 mass% (e.g., 3 to 50 mass%).

The thickness of the shell in the capsule having a core is not particularly limited and may be, for example, 1 to 200 µm, 3 to 150 µm, 5 to 100 µm, etc.

The capsule (e.g., the capsule having a core) may be breakable (disintegrable) (e.g., easily disintegrable or easily breakable). The crush strength of the capsule is determined according to the diameter and other factors, and may be, for example, 100 g or more, 200 g or more, 300 g or more, 400 g or more, 500 g or more, 600 g or more, 700 g or more, 800 g or more, 900 g or more, 1,000 g or more, etc.

The upper limit of the crush strength of the capsule is not particularly specified. The crush strength of the capsule may be, for example, 20,000 g or less, 15,000 g or less, 12,000 g or less, 10,000 g or less, etc.

The crush strength can be measured using, for example, a rheometer (CR-3000EX, manufactured by Sun Scientific).

The ratio of the crush strength (g) to the outer diameter (mm) (crush strength/outer diameter ratio) of the capsule (e.g. , the capsule having a content) is not particularly limited and is, for example, 200 or more (e.g., more than 200), preferably 210 or more (e.g., 220 or more), more preferably 230 or more (e.g., 240 or more); or may be 250 or more, 300 or more, 400 or more, etc.

The upper limit of the ratio of the crush strength to the outer diameter (crush strength/outer diameter ratio) is not particularly specified and may be, for example, 20000, 15000, 10000, 8000, 6000, 5000, etc.

The ratio of the crush strength to the outer diameter can be said to be an index that reflects the practical breakability of the capsule, because there can be cases where the capsule is easily breakable even when the crush strength is high (for example, a case where the outer diameter is long).

The crush deformation of the capsule is determined according to the outer diameter and other factors, and may be, for example, 0.1 mm or more, 0.2 mm or more, 0.5 mm or more, 1.0 mm or more, etc.

The upper limit of the crush deformation of the capsule is not particularly specified. The crush deformation of the soft capsule may be, for example, 15 mm or less, 10 mm or less, 8 mm or less, etc.

The crush deformation can be measured using, for example, a rheometer (CR-3000EX, manufactured by Sun Scientific).

The ratio of the crush deformation (mm) to the outer diameter (mm) (crush deformation/outer diameter ratio) in the capsule is not particularly limited and is, for example, 0.1 or more, preferably 0.12 or more, more preferably 0.15 or more; or may be 0.18 or more, 0.2 or more, etc.

The upper limit of the ratio of the crush deformation to the outer diameter (crush deformation/outer diameter ratio) is not particularly specified and may be, for example, 1.0, 0.98, 0.97, 0.96, 0.95, etc.

The capsule may be used as it is or in combination with another capsule, depending on the application and other factors. The capsule may be, for example, embedded in a filter, as described later.

Said another capsule may be a capsule that does not contain caryophyllene. Said another capsule is, for example, a capsule having a core and a shell in which neither the core nor the shell contains caryophyllene.

Capsules (e.g., seamless capsules) can be produced using known methods. The capsule production methods are described in, for example, Japanese Patent No. 5047285, JP-A H10-506841, Japanese Patent No. 5581446, etc. More specifically, a drop-in-liquid method using a double or multiple nozzle can be employed. In this method, capsule shells are filled with a capsule liquid content, and the shells are then cured and dried to produce seamless capsules.

### Filters

The mode of use of caryophyllene (the agent or composition of the present invention) in filters is not particularly limited. For example, caryophyllene (or the composition) may be contained in (attached to) various parts of a filter (filtration material, filtration member).

In particular, this type of filter may be a capsule-containing filter (a capsule-embedded filter, or a filter composed of a capsule-embedded filtration member).

In other words, this filter contains a capsule containing caryophyllene, which capsule is referred to as a first capsule. The first capsule can be, for example, the same capsule as described in the section “Capsules” above. The first capsule is particularly preferably a capsule having a core and a shell, which core (capsule content) contains caryophyllene.

The filter contains at least the first capsule, and may contain a second capsule, which is different from the first capsule.

The second capsule is not particularly limited as long as it is a capsule different from the first capsule. For example, the second capsule may be a capsule whose content is different from the content of the first capsule.

The second capsule is, for example, a capsule having a core and a shell, which core (and shell) contains a carrier (e.g., a solvent) and/or a flavoring agent (in particular, a capsule that does not contain caryophyllene).

The capsule contained in the above-described type of filter can be the same capsule as described in the section “Capsules” above. The capsule that does not contain caryophyllene (such as the second capsule) can be the same capsule as described in the section “Capsules” above except for the absence of caryophyllene.

The filter is not particularly limited and may be, for example, a filter for air conditioners, air purifiers, etc.

In particular, the capsule-containing filter is suitable for use as a tobacco filter, etc. In a mode of use in tobacco filters, etc., caryophyllene can efficiently be delivered via a pulmonary route and is allowed to efficiently achieve (exert) its functions.

In the case where a capsule containing caryophyllene is embedded in a tobacco filter, a flavoring agent and caryophyllene may be both contained in a single capsule so that users can enjoy both the flavor of the flavoring agent and the effect of the β-caryophyllene; or a flavoring agent and caryophyllene may be contained in separate capsules, both of which are then embedded in the filter, so that users can enjoy both the flavor of the flavoring agent and the effect of the caryophyllene. In the case where a flavoring agent and caryophyllene are contained in separate capsules and used, the following embodiments are conceivable.

Both the capsule containing a flavoring agent and the capsule containing caryophyllene are crushed simultaneously to produce the effects of both capsules simultaneously.

After the capsule containing a flavoring agent is crushed, the capsule containing caryophyllene is crushed.

After the capsule containing caryophyllene is crushed, the capsule containing a flavoring agent is crushed.

### Tobacco Products

The mode of use of caryophyllene (the agent or composition of the present invention) in tobacco products is not particularly limited. For example, caryophyllene (or the composition) may be contained in (attached to) various parts of a tobacco product (tobacco leaf-containing part, tobacco filter, etc.).

Typically and preferably, a capsule or filter containing caryophyllene is used in tobacco products.

As long as the capsule or filter is used in tobacco products, such tobacco products may be conventional tobacco products (combustible tobacco products) or non-combustible tobacco products [e.g., heated tobacco products (direct heated, air heated, etc.)].

### Inhalation Devices

The mode of use of caryophyllene (the agent or composition of the present invention) in inhalation devices is not particularly limited. For example, caryophyllene (or the composition) may be contained in (attached to) various parts of an inhalation device.

The inhalation device is not particularly limited, and examples include smoking devices and non-smoking devices.

Examples of the smoking device include heated tobacco products (such as vapor tobacco products), e-cigarettes, bongs (water pipes), and vaporizers. Heated tobacco products can be used for nicotine intake, while e-cigarettes are nicotine-free. The heated tobacco product is not particularly limited, and examples include IQOS (Philip Morris International), glo (British American Tobacco), Ploom S and Ploom TECH (Japan Tobacco), and Pulze (Imperial Tobacco Group plc). The e-cigarette is not particularly limited, and examples include ego AIO (Joytech) and ICE VAPE (Common Wealth).

The non-smoking device may be, for example, a medical device or a non-medical device (e.g., a health care device). Specific examples of the non-smoking device include inhalers (e.g., nebulizers, steam inhalers), facial machines, and humidifiers.

More specifically, caryophyllene (the agent or composition of the present invention) may be contained in an inhalant in inhalation devices [e.g., a liquid solution in smoking devices such as heated tobacco products (such as vapor tobacco products) and e-cigarettes]. In a mode of use in inhalants, caryophyllene can efficiently be delivered via a pulmonary route and is allowed to efficiently achieve (exert) its functions.

The inhalant (such as a liquid solution) may contain an additional ingredient in addition to caryophyllene. The inhalant usually may contain a carrier [a solvent, a liquid carrier, for example, a polyhydric alcohol (e.g., glycerol, propylene glycol, etc.], and if necessary, may further contain a flavoring agent (may be a flavored liquid solution).

The amount of the caryophyllene in the inhalant (such as a liquid solution) may be selected from the same ranges as described above. For example, the amount of the caryophyllene can be selected from a range of about 0.1 mass% or more (e.g., 0.5 mass% or more) relative to the total mass of the inhalant (such as a liquid solution), and is preferably 1 mass% or more (e.g., 2 mass% or more), more preferably 3 mass% or more (e.g., 5 mass% or more). The amount of the caryophyllene may be 10 mass% or more (e.g., 15 mass% or more, 20 mass% or more, 30 mass% or more, 50 mass% or more, etc.).

In the case where the inhalant contains an additional ingredient, the amount of the additional ingredient is not particularly limited. For example, in the case where the inhalant contains a carrier and/or a flavoring agent, the amount of the carrier and/or the flavoring agent relative to the total mass of the caryophyllene may be selected from the same ranges as described above.

### Perfumery or Cosmetic Products

Perfumery or cosmetic products include air fresheners, oral care products (oral preparations, oral care preparations), cosmetic products, bath salts, perfumes, detergents, fabric softeners, toiletries, pesticides, and paints.

Examples of the air freshener include, but are not particularly limited to, liquid air fresheners and gel air fresheners.

Examples of the oral care product include dentifrices (e.g., toothpaste, gel toothpaste, liquid-state toothpaste, liquid toothpaste, moist toothpaste, etc.), mouthwashes, mouth fresheners, chewing gums, gummies, candies, chocolates, drinks, and tablet confectionaries.

Examples of the cosmetic product include, but are not particularly limited to, basic care products (e.g., lotions, milky lotions, gels, creams, serums, sunscreens, packs, masks, hand creams, body lotions, and body creams), cleansing products (e.g., facial washes, make-up removers, body shampoos, shampoos, conditioners, and treatments), make-up products (e.g., foundations, colors, lipsticks, and lip balms), and hair care products (e.g., tonics, creams, liquids, and sprays). The cosmetic product may be a skin care product.

The mode of use (blending or addition) of caryophyllene (the agent or composition of the present invention) in perfumery or cosmetic products is not particularly limited and can be selected according to the type of perfumery or cosmetic product and other factors. In a mode of use in perfumery or cosmetic products, caryophyllene can efficiently be delivered via a pulmonary route and is allowed to efficiently achieve (exert) its functions.

The amount of the caryophyllene in the perfumery or cosmetic product may be selected from the same ranges as described above.

### Food or Drink Products

Examples of the food or drink product include, but are not particularly limited to, capsules, drinks, foods (processed foods), and confectioneries.

The food or drink product may be, for example, a health food (e.g., a food for specified health uses or a food with nutrient function claims), a dietary supplement, a feed, or a food additive.

Examples of the capsule include, but are not particularly limited to, seamless capsules, soft capsules, and other capsules such as those in the examples above. The shell of the capsule and the embodiment of the capsule (such as a capsule shell) may be as exemplified above.

The mode of use of caryophyllene (the agent or composition of the present invention) in food or drink products is not particularly limited and may be selected according to the embodiment of the food or drink product. For example, in the case of capsules as described above, caryophyllene may be contained in the capsule (e.g., the capsule core and/or shell), or caryophyllene may be added to (blended into) a food or drink product [caryophyllene (the agent or composition of the present invention) may be used as an additive for a food or drink product].

In the case where caryophyllene is added to a food or drink product, examples of the food or drink product include, but are not particularly limited to, foods [e.g., noodles (such as soba, udon, Chinese noodles, and instant noodles), confectioneries, breads, processed seafood or livestock products (such as fish cakes, hams, and sausages), dairy products (such as processed milk and fermented milk), fats or oils and processed fats or oils (such as salad oil, tempura oil, margarine, mayonnaise, shortening, whipped cream, and dressings), seasonings (such as sauces and tare sauces), retort pouch foods (such as curry, stew, rice bowl, porridge, and rice porridge), cold confectioneries (such as ice cream and sherbet), and fried foods]; and drinks (such as tea drinks, soft drinks, carbonated drinks, nutritional drinks, fruit drinks, and lactic acid drinks).

### EXAMPLES

Hereinafter, the present invention will be described in detail (by using Examples and Comparative Examples), but the present invention is not limited thereto.

In the present invention (the EXAMPLES section), β-caryophyllene (INABATA KORYO CO., LTD., Caryophyllene (Caryophyllene AKY-2348)) can be used for purposes including sleep induction or relaxation.

The physical properties of the capsules were measured or evaluated according to the following methods.

### Capsule Crush Strength and Elasticity (Crush Deformation)

The crush strength of the capsules was measured using a rheometer CR-3000EX manufactured by Sun Scientific at room temperature (22 to 27° C.) and 40 to 60% RH.

In the above measurement, the distance by which the capsule deformed until it crushed (the distance by which the capsule was compressed with the rheometer until the capsule crushed) was used as an index of the elasticity of the capsule.

### Capsule Outer Diameter

The outer diameter of the capsules was measured using a digital caliper manufactured by Mitutoyo Corporation (trade name: Quick Mini 25, model number: PK-0510SU, measurement range: 0 to 25 mm) at room temperature (22 to 27° C.) and 40 to 60% RH.

### Capsule Shell Percentage

The percentage of the shell (shell percentage) was calculated as follows: shell percentage (%) = capsule shell mass / total capsule mass × 100.

The mass was measured using an electronic balance GX-200 manufactured by A&D Company, Limited.

### Capsule Shell Thickness

The thickness of the capsule shells (shell thickness) was measured using a digital microscope manufactured by Keyence Corporation (trade name: VHX-900, using a 10 µm calibration scale).

### Experiment A: Measurement of Spatial Concentration Of β-Caryophyllene

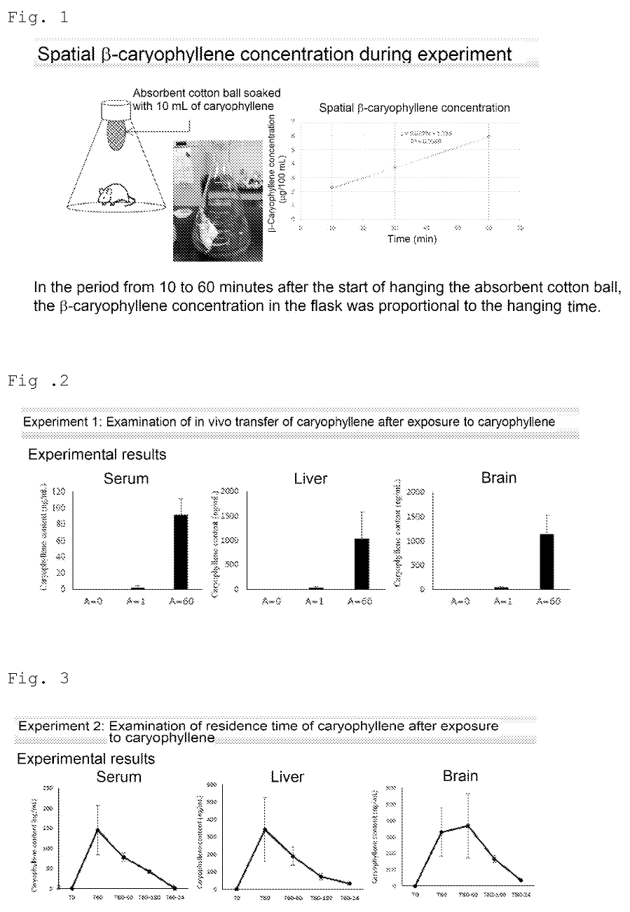

The applicants (present inventors) established an experimental model system using mice. This system enabled mice to inhale spatial concentrations of β-caryophyllene as shown in FIG. 1. Typically, a mouse breathes at a rate of 24 mL/min (Non-Patent Literature 2), meaning that the mouse inhales 1,440 mL of air per 60 minutes. When 10 mL of β-caryophyllene is used in this system, the hourly averaged concentration of β-caryophyllene is about 3.75 µg/100 mL as shown in FIG. 1. Taken together, the amount of the β-caryophyllene delivered pulmonarily to a mouse per hour was roughly estimated to be 54 µg.

Non-Patent Literature 2: Experimental Zoology, detailed exposition, edited by Yoshio Tajima (1972), Asakura Publishing Co., Ltd.

Detailed experimental methods and results are shown below.

Mice were placed in a 5-L flask, and a β-caryophyllene-soaked absorbent cotton ball was hung at the top of the flask so that the mice could inhale β-caryophyllene.

### Experimental Method

An absorbent cotton ball soaked with 10 mL, 5 mL, 0.5 mL, or 0.05 mL of β-caryophyllene was hung at the top of a 5-L flask so that the β-caryophyllene was allowed to volatilize for a certain period of time. Afterwards, 200 mL of the volatiles were adsorbed on the adsorbent InertSep C18 (200 mg/1 mL) using a minipump MP-∑NII. The adsorbed aroma ingredient was eluted with 1 mL of a solvent (methanol) , and the resulting eluate was made up to a total volume of 1 mL. Then, 1 µL of the sample was injected into a GC/MS system for quantification analysis. The quantified amount of β-caryophyllene was converted into the amount per 100 mL of air.

### Experimental Results

The spatial concentration (µg/100 mL) in the flask is shown in Table 1.

TABLE 1

The concentration of β-caryophyllene volatilized in a flask for a certain period of time

| | 10 min | 30 min | 60 min | Average |
|---|---|---|---|---|
| 10 mL | 2.80±0.24 | 4.33±0.07 | 4.96±0.08 | 4.03 |
| 5 mL | 1.51±0.13 | 2.84±0.17 | 3.40±0.30 | 2.58 |
| 0.5 mL | 0.620±0.061 | 1.153±0.020 | 1.380±0.035 | 1.05 |
| 0.05 mL | 0.352±0.018 | 0.482±0.003 | 0.673±0.006 | 0.502 |

Since a mouse breathes at a rate of 24 mL/min (Non-Patent Literature 2), meaning that the mouse inhales 1,440 mL of air per 60 minutes, the amount of the β-caryophyllene delivered pulmonarily to a mouse per hour can be given the values shown in Table 2.

TABLE 2

The amount of β-caryophyllene delivered pulmonarily to a mouse per hour

| | Amount delivered pulmonarily (µg) |
|---|---|
| 10 mL | 58 |
| 5 mL | 37 |
| 0.5 mL | 15 |
| 0.05 mL | 7.2 |

Generally, the effects of β-caryophyllene correlate with its blood level, and the blood level is proportional to the amount delivered per body weight. A mouse weighs about 20 g, and a human weighs about 70 kg on average. Therefore, the amount of inhaled β-caryophyllene in a human to achieve its blood level corresponding to that in a mouse is presumably 3,500 times greater than that in a mouse.

### Experiment 1: Bioavailability of β-Caryophyllene Delivered by Inhalation

Four-week-old Slc:ddY mice were purchased from Shimizu Laboratory Supplies, Co., Ltd. and housed at a room temperature of 25 ± 1° C. on a 12-hour light/dark cycle. After 5 days of habituation, the mice were divided into experimental groups.

The mice were exposed to β-caryophyllene inhalation using the device shown in FIG. 1. After that, the mice were anesthetized and dissected to collect the whole brain (cerebrum and cerebellum), liver (entire left lobe), and blood (about 1 mL). Each organ was ground in a mortar and subjected to extraction of β-caryophyllene using acetone. The extracts were volatilized, adsorbed on a Tenax tube (GERSTEL K.K. Japan, TDU tube Tenax TA), and analyzed with a GC/MS system (Agilent Technologies, Inc., 7890B/5977B GC/MSD) for concentration quantification.

Nine mice were divided into 3 groups. Three mice were assigned to a 0-min caryophyllene exposure group (A=0 group), 3 mice were assigned to a 1-min caryophyllene exposure group (A=1 group) , and 3 mice were assigned to a 60-min caryophyllene exposure group (A=60 group). An absorbent cotton ball soaked with 10 mL of β-caryophyllene was hung on the top of a 1-L flask as shown in FIG. 1 and left to stand for 10 minutes so that the flask was filled with β-caryophyllene. The mice in the A=0 group were not placed into the flask, and dissection was started 10 minutes after anesthesia. The mice in the A=1 group were placed in the flask for one minute, taken out from the flask, and anesthetized. The mice in the A=60 group were placed in the flask for 60 minutes, taken out from the flask, and anesthetized.

As shown in FIG. 2, the β-caryophyllene was sufficiently transferred to the serum, liver, and brain after the 60-minute inhalation of the β-caryophyllene present in the space in the flask.

### Experiment 1A: Bioavailability of β-Caryophyllene Delivered by Inhalation

### Experimental Method

The same experiment as described in Experiment 1 was performed.

Four-week-old Slc:ddY mice were purchased from Shimizu Laboratory Supplies, Co., Ltd. and housed at a room temperature of 25 ± 1° C. on a 12-hour light/dark cycle. After 5 days of habituation, the mice were divided into experimental groups.

The mice were exposed to β-caryophyllene inhalation using the same device as above. After that, the mice were anesthetized and dissected to collect the whole brain (cerebrum and cerebellum), liver (entire left lobe), and blood (about 1 mL). Each organ was ground in a mortar and subjected to extraction of β-caryophyllene using acetone. The extracts were volatilized, adsorbed on a Tenax tube (GERSTEL K.K. Japan, TDU tube Tenax TA), and analyzed with a GC/MS system (Agilent Technologies, Inc., 7890B/5977B GC/MSD) for concentration quantification.

Three mice were divided into 3 groups. One mouse was assigned to a 0-min caryophyllene exposure group, one mouse was assigned to a 1-min caryophyllene exposure group, and one mouse was assigned to a 60-min caryophyllene exposure group. An absorbent cotton ball soaked with 10 mL of β-caryophyllene was hung on the top of a 1-L flask as shown in FIG. 1 and left to stand for 10 minutes so that the flask was filled with β-caryophyllene. The mouse in the 0-min caryophyllene exposure group was not placed into the flask, and dissection was started 10 minutes after anesthesia. The mouse in the 1-min caryophyllene exposure group was placed in the flask for one minute, taken out from the flask, and anesthetized. The mouse in the 60-min caryophyllene exposure group was placed in the flask for 60 minutes, taken out from the flask, and anesthetized.

### Experimental Results

As shown in Table 3, the β-caryophyllene was transferred to the serum, liver, and brain after the 60-minute inhalation of the β-caryophyllene present in the space in the flask.

TABLE 3

The concentrations of β-caryophyllene in the serum, liver, and brain of a mouse exposed to β-caryophyllene for 0, 1, or 60 minutes

| | | | |
|---|---|---|---|
| | 0-min exposure | 1-min exposure | 60-min exposure |
| Serum | 0 ng/mL | 1.6 ng/mL | 102 ng/mL |
| Liver | 0 ng/g | 28 ng/g | 1127 ng/g |
| Brain | 0 ng/g | 44 ng/g | 1325 ng/g |

### Experiment 1B: Bioavailability of β-Caryophyllene Delivered by Inhalation or Oral Administration

### Experimental Method

β-Caryophyllene was orally administered to mice using a sonde at a dose of 20 µg per gram of animal body weight. The amount of the β-caryophyllene delivered to a mouse weighing 25 g was 500 µg. Since the β-caryophyllene delivered pulmonarily to a mouse per hour is estimated to be 54 µg based on the results of Experiment A1, the amount of the β-caryophyllene orally administered is about 10 times the amount delivered pulmonarily.

Three mice were divided into the following three groups: a β-caryophyllene non-administration group, a 60-min β-caryophyllene exposure group, and a 20 µg/g β-caryophyllene oral administration group. After grouping, the concentrations of β-caryophyllene in the serum, thoracic aorta, and abdominal aorta were determined in the same manner as described above. The mouse in the oral administration group was dissected 30 minutes after oral administration to collect the serum, thoracic aorta, and abdominal aorta.

### Experimental Results

TABLE 4

The concentrations of β-caryophyllene in the serum, thoracic aorta, and abdominal aorta of a mouse subjected to pulmonary delivery or oral administration of β-caryophyllene

| | | | |
|---|---|---|---|
| | Control | 60-min exposure | Oral administration |
| Serum | 0 ng/mL | 34 ng/mL | 8.8 ng/mL |
| Thoracic aorta | 0 ng/g | 751 ng/g | 1322 ng/g |
| Abdominal aorta | 0 ng/g | 403 ng/g | 210 ng/g |

As shown in Table 4, there was an about 4-fold or less difference in the β-caryophyllene concentrations in the serum, thoracic aorta, and abdominal aorta between the 60-min β-caryophyllene exposure group and the β-caryophyllene oral administration group. Considering that oral administration provides about 10-fold amount of β-caryophyllene as compared with pulmonary delivery, the bioavailability of the latter is higher.

### Experiment 2: Pharmacokinetics of β-Caryophyllene

If β-caryophyllene resides in the body for a long period of time, it may adversely affect physiological functions because of its unknown collateral effects. The following experiment was performed to determine the residence time of β-caryophyllene in the body.

Thirty-one mice were divided into 5 groups. Seven mice were assigned to a 0-min caryophyllene exposure group (T0), 6 mice were assigned to a 60-min caryophyllene exposure group (T60), 6 mice were assigned to a 60-min caryophyllene exposure and 60-min normal breathing group (T60-60), 6 mice were assigned to a 60-min caryophyllene exposure and 180-min normal breathing group (T60-180), and 6 mice were assigned to a 60-min caryophyllene exposure and 24-hour normal breathing group (T60-24). After grouping, the organ-specific concentrations of β-caryophyllene were determined in the same manner as described in Experiment 1.

As shown in FIG. 3, in a period when the mice were kept in a clean air environment after a 60-minute inhalation of caryophyllene, the concentrations of β-caryophyllene in highly water-soluble organs, such as the serum and liver, were markedly decreased after 3 hours. On the other hand, the concentration of β-caryophyllene in the brain was increased after 3 hours, but markedly decreased after 24 hours. These results show that β-caryophyllene is moderately metabolized and eliminated without excessive accumulation in the body, indicating that β-caryophyllene is highly safe.

### Experiment 2A: Pharmacokinetics of β-Caryophyllene Experimental Method

The same experiment as described in Experiment 2 was performed.

Five mice were divided into 5 groups. One mouse was assigned to a 0-min caryophyllene exposure group, one mouse was assigned to a 60-min caryophyllene exposure group, one mouse was assigned to a 60-min caryophyllene exposure and 60-min normal breathing group, one mouse was assigned to a 60-min caryophyllene exposure and 180-min normal breathing group, and one mouse was assigned to a 60-min caryophyllene exposure and 24-hour normal breathing group. After grouping, the organ-specific concentrations of β-caryophyllene were determined in the same manner as described in Experiments 1 and 1A.

### Experimental Results

As shown in Table 5, in a period when the mice were kept in a clean air environment after a 60-minute inhalation of caryophyllene, the concentrations of β-caryophyllene in highly water-soluble organs, such as the serum and liver, were markedly decreased after 3 hours. On the other hand, the concentration of β-caryophyllene in the brain was increased after 3 hours, but markedly decreased after 24 hours. These results show that β-caryophyllene is moderately metabolized and eliminated without excessive accumulation in the body, indicating that β-caryophyllene is highly safe.

TABLE 5

Table 5: The concentrations of β-caryophyllene in the serum, liver, and brain of a mouse kept in a clean air environment for 60 minutes, 120 minutes, or 24 hours after 60-min β-caryophyllene exposure

| | | | | | |
|---|---|---|---|---|---|
| | 0-min exposure | 60-min exposure | 60-min exposure and 60-min normal breathing | 60-min exposure and 180-min normal breathing | 60-min exposure and 24-hour normal breathing |
| Serum | 0 ng/mL | 142 ng/mL | 77 ng/mL | 42 ng/mL | 0 ng/mL |
| Liver | 0 ng/g | 298 ng/g | 170 ng/g | 69 ng/g | 33 ng/g |
| Brain | 0 ng/g | 283 ng/g | 307 ng/g | 172 ng/g | 37 ng/g |

### Experiment 3: Relaxing and Sleep-Inducing Effects of Inhaled β-Caryophyllene

In the present invention (this experiment), the relaxing effect is defined as significantly increasing the motionless time in comparison with a control group, and similarly, the sleep-inducing effect is defined as significantly increasing the sleep time in comparison with a control group.

Animal experiments were performed as follows. Eight mice were divided into two groups. Four mice were assigned to a control group (60-min normal breathing in a flask) , and 4 mice were assigned to a caryophyllene group (60-min exposure to caryophyllene) . The motionless time and the sleep time of the mice were measured as follows. The mice were placed in 1-L flasks, and their behaviors were monitored over 1 hour. During the monitoring, the period of a state in which a mouse was kept motionless for one second or more was measured, and the cumulative period was regarded as the motionless time. During the monitoring, the period of a state in which a mouse closed its eyes for one second or more was measured, and the cumulative period was regarded as the sleep time.

### Example 1

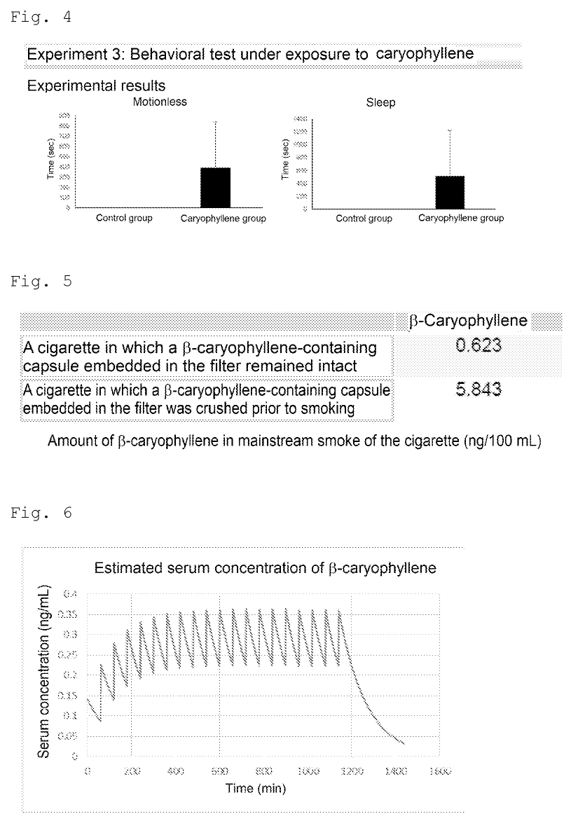

Mice were placed in a flask filled with β-caryophyllene, and the motionless time and the sleep time were measured. As shown on the right in each graph in FIG. 4, the motionless time was 390 seconds, and the sleep time was 512 seconds.

### Comparative Example 1

Mice were placed in a flask filled with clean air, and the motionless time and the sleep time were measured. As shown on the left in each graph in FIG. 4, the motionless time was 0 second, and the sleep time was 0 second.

### Experiment 3A: Relaxing and Sleep-Inducing Effects of Inhaled β-Caryophyllene

The same experiment as described in Experiment 3 was performed.

### Experimental Method

Five mice were divided into five groups. One mouse was assigned to a 5-mL caryophyllene group (Example 3A-1) , one mouse was assigned to a 0.5-mL caryophyllene group (Example 3A-2) , one mouse was assigned to a 0.05-mL caryophyllene group (Example 3A-3) , and one mouse was assigned to a control group (Comparative Example 3A-1). The motionless time and the sleep time of the mice were measured as follows . Individual mice were placed in separate 5-L flasks, and their behaviors were monitored over 1 hour. A state in which a mouse did not move for 30 seconds or more was regarded as “motionless”, and the time to first motionless period was measured as the time to motionlessness onset, and the cumulative motionless period was measured as the motionless time. A state in which a mouse did not move with eyes being at least two-third closed was regarded as “sleep”, and the time to first sleep period was measured as the time to sleep onset, and the cumulative sleep period was measured as the sleep time.

### Experimental Results

As shown in Table 6, relaxing effect was observed at any amount of β-caryophyllene absorbed in the absorbent cotton ball, and the most relaxing effect was observed at an amount of 0.5 mL. Similarly, sleep inducing effect was observed at any amount of β-caryophyllene absorbed in the absorbent cotton ball, and the most sleep-inducing effect was observed at amount of 0.5 mL.

TABLE 6

Relaxing and sleep-inducing effects of inhaled β-caryophyllene in mice

| | | | | | |
|---|---|---|---|---|---|
| | Amount of β-caryophyllene absorbed in absorbent cotton ball | Time to motionlessness onset | Motionless time | Time to sleep onset | Sleep time |
| Example 3A-1 | 5 mL | 318 sec | 2626 sec | 2660 sec | 1474 sec |
| Example 3A-2 | 0.5 mL | 220 sec | 3111 sec | 975 sec | 2701 sec |
| Example 3A-3 | 0.05 mL | 297 sec | 2686 sec | 2329 sec | 1043 sec |
| Comparative Example 3A-1 | 0 mL | 769 sec | 1774 sec | 3470 sec | 245 sec |

### Experiment 4: Encapsulation of β-Caryophyllene

The above test confirmed sleep-inducing and relaxing effects of β-caryophyllene using an absorbent cotton ball. In the present invention (this experiment), the applicants found that efficient inhalation of β-caryophyllene can be achieved by using a seamless capsule containing β-caryophyllene, more specifically, breaking such a seamless capsule in a tubular filter to allow the content to diffuse out of the capsule.

β-Caryophyllene, which was the same as that used in the above animal experiments, was entrapped into a seamless capsule by a drop method. The seamless capsule had a diameter of 3.35 mm (about 3.4 mm) and a liquid content mass of 19.3 mg. This type of capsule is the same as the type of capsule prepared in Example 5-1 below.

The seamless capsule containing the above composition in the shell was embedded in an acetate filter of a cigarette. The seamless capsule was crushed just before use, and the cigarette was lit and smoked in a smoking machine. FIG. 5 shows the spatial concentration of β-caryophyllene in smoking the cigarette. The smoking machine used was Linear Smoking Machine (LM2) manufactured by Borgwaldt GMBH. The mainstream smoke was collected in a gas bag (GL Sciences Inc., Odor bag 3 L) according to the method specified in ISO 3308. After that, 200 mL of the sampled mainstream smoke was adsorbed on a Tenax tube (GERSTEL K.K. Japan, TDU tube Tenax TA) using a sampling pump (GASTEC CORPORATION, gas sampling pump model 801) and analyzed with a GC/MS system (Agilent Technologies, Inc., 7890B/5977B GC/MSD) for concentration quantification.

In this way, volatilization and inhalation of caryophyllene from the above composition can be achieved.

### Example 2

A capsule containing 20 µL of β-caryophyllene was prepared and embedded in a cigarette filter. The capsule was broken just before use, and the cigarette was lit and smoked. The spatial concentration of β-caryophyllene in smoking the cigarette was measured (FIG. 5).

As shown in FIG. 5, 0.623 ng/100 mL of β-caryophyllene was detected in smoking a lit cigarette in which a capsule containing 20 µL of β-caryophyllene was embedded in the cigarette filter. On the other hand, in the case of breaking the capsule before lighting the cigarette, the amount of β-caryophyllene detected was 5.843 ng/100 mL, which was significantly higher than that without breaking the capsule.

Since the density of air is 1.293 kg/m³, when the concentration of β-caryophyllene in air is 3.75 µg/100 mL, the mass ratio of β-caryophyllene to air is 3.75 µg/0.129 g, that is, the concentration of caryophyllene in air is 0.0029%.

Next, consider the concentration of β-caryophyllene at equilibrium in air when β-caryophyllene is diffused in air. To this end, the value of the vapor pressure of β-caryophyllene at 25° C. needs to be determined. Assuming that the molar enthalpy of vaporization is not affected by temperature, the Clausius-Clapeyron equation expressing the relationship between vapor pressure and temperature can be rewritten as:

$${p}_{v a p} = {p}_{0} exp \left[\frac{{Δ}_{v a p} {H}_{m}}{R} \left(\frac{1}{{T}_{0}} - \frac{1}{T}\right)\right] .$$

The molar enthalpy of vaporization (ΔvapHm) for water is 44.0 kJ/mol. The molar enthalpy of vaporization (ΔvapHm) for β-caryophyllene is not reported, but considering that β-caryophyllene and octane are both hydrocarbon molecules and have similar boiling points, the molar enthalpy of vaporization for β-caryophyllene is presumed to be equivalent to the molar enthalpy of vaporization for octane, which is 35.0 kJ/mol. The boiling point of octane is 125.7° C., and the boiling point of β-caryophyllene is 130° C. Substituting T₀ = 403 K (130° C.) and p₀ = 1.0 × 10⁵ Pa, which is atmospheric pressure, into the above equation gives a value of the vapor pressure of β-caryophyllene at 27° C., which is 2.5 × 10³ Pa. Therefore, when β-caryophyllene is abundantly present, the concentration of β-caryophyllene at equilibrium is 2.8 × 10³ Pa/1.0 × 10⁵ Pa = 2.8%.

The evaporation rate of β-caryophyllene is presumably proportional to the difference between the concentration of β-caryophyllene at equilibrium and the concentration of β-caryophyllene in air. Therefore, the evaporation rate of β-caryophyllene can be expressed as an exponential function that converges to the concentration of β-caryophyllene at equilibrium.

The exponential function presumably represents a proportional relationship in the t = 0 limit as seen in FIG. 1. Considering that the 2.5% β-caryophyllene is equivalent to 3,232 µg/100 mL and that the slope in the t = 0 limit is 0.0739 [µg/100 mL/min], the volatilization rate of caryophyllene in the case of hanging an absorbent cotton ball soaked with 10 mL of β-caryophyllene is expressed by:

$$\text{y} \text{=} \text{3232} \times \left(1 - {e}^{- \frac{t}{48985}}\right) .$$

The unit of y is µg/100 mL, and the unit of t is minutes. The half-life of this exponential function is 23.5 days, indicating that the volatilization rate of caryophyllene at room temperature is very slow.

In the case of crushing a seamless capsule, the spatial concentration of volatilized β-caryophyllene is as follows. An example is given here where the amount of β-caryophyllene contained in the seamless capsule is 20 µL. Generally, the volatilization rate of β-caryophyllene is proportional to the surface area and the surface area is proportional to the ⅔ power of the volume. Therefore, in the case where the β-caryophyllene-containing seamless capsule is crushed in a 1-L space, its spatial concentration is expressed by:

$$\text{y = 3232} \times \left(1 - {e}^{- {\left(\frac{20}{10 \times {10}^{5}}\right)}^{\frac{2}{3}} \times \frac{t}{48985}}\right) = 3232 \times \left(1 - {e}^{- \frac{t}{3085862}}\right) .$$

At least in the period from 0 to 10 minutes after start, the concentration of β-caryophyllene is proportional to time and thus expressed by y = 0.001 × t. The unit of y is µg/100 mL, and the unit of t is minutes. For example, the concentration of β-caryophyllene at one minute after start is 1 ng/100 mL. The concentration is constant regardless of the volume of the space.

On the other hand, in the above Example, a 2-second air suction (1.05 L/min) was repeated 8 times at 58-second intervals. The total time for exposure to fresh air was 16 seconds, during which the concentration became 5.8 ng/100 mL. This is 21 times more efficient than the former.

To summarized the above, encapsulation of β-caryophyllene allows inhalation of a higher concentration of β-caryophyllene, providing advantages in terms of relaxing effects and others.

### Other Embodiments

The above embodiments illustrate a case where a gelatin-free capsule coat (shell) is used for encapsulation, but this is a non-limiting example. It will be understood that a gelatin-containing capsule coat can be used as well.

The above embodiments mainly illustrate a case where the capsule is embedded in a tobacco filter, but this is a non-limiting example. Any mode of use may be employed as long as β-caryophyllene can be pulmonarily delivered. For example, β-caryophyllene can be pulmonarily delivered using an inhalation device that allows the inhalation of volatilized β-caryophyllene without combustion, unlike tobacco products, by which β-caryophyllene is pulmonarily delivered with smoke.

In addition, another example is so-called flavored tobacco products, in which a flavoring agent is encapsulated and embedded in a filtration member of tobacco products. In the case where a capsule containing β-caryophyllene is embedded in the filter of flavored tobacco products, a flavoring agent and β-caryophyllene may be both contained in a single capsule so that users can enjoy both the flavor of the flavoring agent and the effect of the β-caryophyllene; or a flavoring agent and β-caryophyllene may be contained in separate capsules, both of which are then embedded in the filter, so that users can enjoy both the flavor of the flavoring agent and the effect of the β-caryophyllene. In the case where a flavoring agent and β-caryophyllene are contained in separate capsules (that is, the first capsule containing β-caryophyllene and the second capsule at least containing a flavoring agent differ in their liquid content) and used, the embodiments described below are conceivable. It should be noted that the term “differ” here includes not only cases where all of the ingredients and their compositions are completely different, but also cases where some of the ingredients are different. The concept of “differ” here shall also include cases where the ingredients are the same but the composition ratios are different.

Both the capsule containing a flavoring agent and the capsule containing β-caryophyllene are crushed simultaneously to produce the effects of both capsules simultaneously.

After the capsule containing a flavoring agent is crushed, the capsule containing β-caryophyllene is crushed.

After the capsule containing β-caryophyllene is crushed, the capsule containing a flavoring agent is crushed.

It will be understood that the liquid content of the second capsule can contain a flavoring agent alone or in combination with an oily ingredient or other ingredients.

### Experiment 5: Amount of Volatilized β-Caryophyllene in Smoking a Cigarette Containing a β-Caryophyllene Capsule in the Cigarette Filter

### Sample Preparation

The cigarette used was CORESTA Monitor 9 (CM9) , which was purchased from Borgwaldt GMBH.

The β-caryophyllene used was Caryophyllene AKY-2348, which was purchased from INABATA KORYO CO., LTD.

The 1-menthol used was a recrystallized product from steam-distilled essential oil from Mentha canadensis, which product was purchased from Anhui Tonghui Perfume Co., Ltd.

The MCT used was a pressed oil product from Elaeis guineensis fruits, which product was purchased from Kao Corporation.

A spearmint flavoring agent with 15% β-caryophyllene (mass%, hereinafter the same in the compositions) was prepared by blending a steam-distilled essential oil product from Mentha spicata with β-caryophyllene at a final concentration of 15%.

An apple flavoring agent type 1 with 15% β-caryophyllene was prepared by blending hexanol, hexanal, 2-methylbutyl hexanoate, hexyl acetate, and hexyl hexanoate as main ingredients and further blending this mixture with β-caryophyllene at a final concentration of 15%.

A grape flavoring agent with 15% β-caryophyllene was prepared by blending dimethyl anthranilate, ethyl acetate, ethyl propionate, styralyl acetate, propionic acid, ethyl maltol, cis-3-hexenol, β-ionone, raspberry ketone, and methyl isoeugenol as main ingredients and further blending this mixture with β-caryophyllene at a final concentration of 15%.

A mango flavoring agent with 15% β-caryophyllene was prepared by blending Caryophyllene AKY-2348 (15%), Mango base AKY-2750 (35%) , both of which were purchased from INABATA KORYO CO., LTD., and MCT (50%).

A blueberry flavoring agent with 15% β-caryophyllene was prepared by blending Caryophyllene AKY-2348 (15%), Blueberry concentrate 10X AKY-2896 (10%), both of which were purchased from INABATA KORYO CO., LTD., and MCT (75%).

An apple flavoring agent type 2 with 15% β-caryophyllene was prepared by blending Caryophyllene AKY-2348 (15%), Apple base AKY-2712 (35%), both of which were purchased from INABATA KORYO CO., LTD., and MCT (50%).

A chamomile tea flavoring agent with 15% β-caryophyllene was prepared by blending Caryophyllene AKY-2348 (15%), Chamomile tea AKY-2845 (35%), both of which were purchased from INABATA KORYO CO., LTD., and MCT (50%).

A green tea flavoring agent with 15% β-caryophyllene was prepared by blending Caryophyllene AKY-2348 (15%), Green tea flavor AKY-1871 (10%), both of which were purchased from INABATA KORYO CO., LTD., and MCT (75%).

A lemon flavoring agent with 15% β-caryophyllene was prepared by blending Caryophyllene AKY-2348 (15%), Citrus concentrate 5XAKY-2745 (20%), both of which were purchased from INABATA KORYO CO., LTD., and MCT (65%).

### Experimental Method

The 12 types of easily disintegrable capsules described below were prepared by a drop method. The capsule diameter was 3.4 mm (shell thickness: 50 µm, liquid content mass: 19.3 mg) . For the capsule shell, agar, hydrolyzed guar gum, sodium alginate, carrageenan, dextrin, glycerol, and dye were dissolved in water to form a sol solution, and the resulting sol solution was used. The sol solution was composed of 2.7 mass% agar, 1.9 mass% hydrolyzed guar gum, 1.9 mass% sodium alginate, 0.7 mass% carrageenan, 0.1 mass% dextrin, 0.7 mass% glycerol, 0.02 mass% dye, and water (balance).

The capsules had a crush strength of 153 g and a crush deformation of 1.4 mm.

The compositions of the liquid contents are as follows.

Example 5-1: 100% β-Caryophyllene
Example 5-2: 15% β-Caryophyllene, 15% L-menthol, and MCT 70%.
Example 5-3: Spearmint flavoring agent with 15% β-caryophyllene
Example 5-4: Apple flavoring agent type 1 with 15% β-caryophyllene
Example 5-5: Grape flavoring agent with 15% β-caryophyllene
Example 5-6: Mango flavoring agent with 15% β-caryophyllene
Example 5-7: Blueberry flavoring agent with 15% β-caryophyllene
Example 5-8: Apple flavoring agent type 2 with 15% β-caryophyllene
Example 5-9: Chamomile tea flavoring agent with 15% β-caryophyllene
Example 5-10: Green tea flavoring agent with 15% β-caryophyllene
Example 5-11: Lemon flavoring agent with 15% β-caryophyllene
Comparative Example 5-1: 100% MCT

In addition to capsules having a capsule diameter of 3.4 mm, the easily disintegrable capsules described below were also prepared by a drop method. The formula of the capsule shell was as described above.

Example 5-12: Capsule diameter: 2.8 mm, shell thickness: 57 µm, liquid content formula: 15% β-caryophyllene, 15% L-menthol, and 70% MCT (liquid content mass: 10 mg), crush strength: 118 g, crush deformation: 1.5 mm
Example 5-13: Capsule diameter: 3.0 mm, shell thickness: 48 µm, liquid content formula: 15% β-caryophyllene, 15% L-menthol, and 70% MCT (liquid content mass: 13 mg), crush strength: 127 g, crush deformation: 1.6 mm
Example 5-14: Capsule diameter: 3.5 mm, shell thickness: 48 µm, liquid content formula: 15% β-caryophyllene, 15% L-menthol, and 70% MCT (liquid content mass: 20 mg), crush strength: 167 g, crush deformation: 1.8 mm
Example 5-15: Capsule diameter: 4.0 mm, shell thickness: 45 µm, liquid content formula: 15% β-caryophyllene, 15% L-menthol, and 70% MCT (liquid content mass: 34 mg), crush strength: 206 g, crush deformation: 2.0 mm

In addition, the easily disintegrable capsules of 3.4 mm in diameter described below, having different concentrations of β-caryophyllene in the liquid content, were prepared by a drop method.

Example 5-16: 5% β-Caryophyllene, 15% L-menthol, and 80% MCT.

Example 5-17: 10% β-Caryophyllene, 15% L-menthol, and 75% MCT.

Example 5-18: 30% β-Caryophyllene, 15% L-menthol, and 55% MCT.

Example 5-19: 50% β-Caryophyllene, 15% L-menthol, and 35% MCT.

Example 5-20: 15% β-Caryophyllene, 35% L-menthol, and 50% MCT.

Example 5-21: 15% β-Caryophyllene, 45% L-menthol, and 40% MCT.

The capsules prepared in the above Examples and Comparative Example were inserted in the center of cigarette filters. The smoking machine used was Linear Smoking Machine (LM2) manufactured by Borgwaldt GMBH. Smoking was performed according to the method specified in ISO 3308 (35 mL puff of 2-second duration taken per minute). Vapor and particulate ingredients of three cigarettes were adsorbed on a glass filter of the smoking machine. The amount of volatilized β-caryophyllene per cigarette was measured by GC/MS based on the method specified in ISO 10315.

### Experimental Results

TABLE 7

Mass of β-caryophyllene in mainstream smoke in smoking one lit cigarette in which a β-caryophyllene-containing capsule embedded in the cigarette filter was broken

| | | |
|---|---|---|
| | | Amount of β-caryophyllene |
| Example 5-22 | A cigarette in which the capsule of Example 5-1 embedded in the filter was broken prior to smoking. | 2.99 mg |
| Example 5-23 | A cigarette in which the capsule of Example 5-2 embedded in the filter was broken prior to smoking. | 0.29 mg |
| Example 5-24 | A cigarette in which the capsule of Example 5-3 embedded in the filter was broken prior to smoking. | 0.90 mg |
| Example 5-25 | A cigarette in which the capsule of Example 5-4 embedded in the filter was broken prior to smoking. | 0.99 mg |
| Example 5-26 | A cigarette in which the capsule of Example 5-5 embedded in the filter was broken prior to smoking. | 1.33 mg |
| Comparative Example 5-2 | A cigarette in which the capsule of Comparative Example 5-1 embedded in the filter was broken prior to smoking. | Below detection limit |

In Example 5-22, about 3 mg of β-caryophyllene was detected in smoking a lit cigarette in which a capsule containing β-caryophyllene had been embedded in the cigarette filter. In Examples 5-23 to 5-26, about 0.3 to 1.3 mg of β-caryophyllene was detected in smoking a lit cigarette in which a capsule containing β-caryophyllene had been embedded in the cigarette filter. In this way, volatilization and inhalation of caryophyllene from the above composition can be achieved.

In this experiment, direct adsorption on the glass filter significantly increased the amount of β-caryophyllene detected compared to that in Experiment 4, which amount accurately reflects the actual amount of inhaled β-caryophyllene.

### Experiment 6: Amount of Volatilized β-Caryophyllene in Vaping E-Cigarettes Containing β-Caryophyllene

### Experimental Method

The e-cigarette used was ICE VAPE X-TC1 purchased from Shenzhen Joecig Technology Co., Ltd.

A composition (flavored liquid solution) was prepared as follows.

The sucrose fatty acid ester used was purchased from DKS Co., Ltd.

Flavored liquid solution 1: 5 parts by mass of propylene glycol, 4.5 parts by mass of glycerol, 0.5 part by mass of P-caryophyllene, and 0.01 part by mass of sucrose fatty acid ester.

In addition, other compositions (flavored liquid solutions) were prepared based on the ingredient ratios shown in Table 8 below.

TABLE 8

Flavored liquid solutions

| | Propylene glycol | Glycerol | β-Caryophyhene | Flavoring agent |
|---|---|---|---|---|
| Flavored liquid solution 2 | 46% | 44% | 5% | 5% Lemon flavoring agent |
| Flavored liquid solution 3 | 46% | 44% | 5% | 5% Apple flavoring agent |
| Flavored liquid solution 4 | 44% | 41% | 5% | 10% Black tea flavoring agent |
| Flavored liquid solution 5 | 44% | 41% | 5% | 10% Green tea flavoring agent |
| Flavored liquid solution 6 | 49% | 46% | 0% | 5% Lemon flavoring agent |
| Flavored liquid solution 7 | 49% | 46% | 0% | 5% Apple flavoring agent |
| Flavored liquid solution 8 | 47% | 43% | 0% | 10% Black tea flavoring agent |
| Flavored liquid solution 9 | 47% | 43% | 0% | 10% Green tea flavoring agent |

The prepared flavored liquid solutions were injected into the ICE VAPE X-TC1. The smoking machine used was Linear Smoking Machine (LM2) manufactured by Borgwaldt GMBH. Vaping was performed according to the method specified in ISO 3308. Eight puffs were taken per e-cigarette. Vapor and particulate ingredients of three cigarettes were adsorbed on a glass filter of the smoking machine. The amount of volatilized β-caryophyllene per cigarette was measured by GC/MS based on the method specified in ISO 10315.

### Experimental Results

TABLE 9

Mass of β-caryophyllene in mainstream smoke in vaping one e-cigarette in which a β-caryophyllene-containing flavored liquid solution was injected

| | | |
|---|---|---|
| | | Amount of β-caryophyllene |
| Example 6-1 | Flavored liquid solution 1 | 0.54 mg |
| Example 6-2 | Flavored liquid solution 2 | 0.41 mg |
| Example 6-3 | Flavored liquid solution 3 | 0.29 mg |
| Example 6-4 | Flavored liquid solution 4 | 0.55 mg |
| Example 6-5 | Flavored liquid solution 5 | 0.80 mg |
| Comparative Example 6-1 | Flavored liquid solution 6 | Below detection limit |
| Comparative Example 6-2 | Flavored liquid solution 7 | Below detection limit |
| Comparative Example 6-3 | Flavored liquid solution 8 | Below detection limit |
| Comparative Example 6-4 | Flavored liquid solution 9 | Below detection limit |

As shown in Table 9, in Examples 6-1 to 6-5, about 0.3 to 0.8 mg of β-caryophyllene was detected. In this way, volatilization and inhalation of caryophyllene from the above composition can be achieved.

### Experiment 7: Capsules Containing β-Caryophyllene Sample Preparation

The gelatin used was purchased from Nitta Gelatin Inc.

### Experimental Method

The capsules described below were prepared by a drop method. The capsule diameter was 5.0 mm (shell thickness: 118 µm, liquid content mass: 55.6 mg). For the capsule shell, gelatin and glycerol were dissolved in water to form a sol solution, and the resulting sol solution was used. The sol solution was composed of 20.8 mass% gelatin, 4.2 mass% glycerol, and 75.0 mass% water.

The composition of the liquid content is as follows.

Example 7-1: 15% β-Caryophyllene and 85% MCT.

Example 7-2: 25% β-Caryophyllene and 75% MCT.

Example 7-3: 50% β-Caryophyllene and 50% MCT.

Comparative Example 7-1: 100% MCT.

By ingesting any of the above capsules, oral delivery of β-caryophyllene was achieved.

### Experiment 8: Oral Compositions Containing β-Caryophyllene Sample Preparation

Polyoxyethylene (20) stearyl ether, xylitol, tocopherol acetate, and propylene glycol were purchased from FUJIFILM Wako Pure Chemical Corporation.

Xanthan gum and sodium alginate were purchased from KIMICA Corporation.

### Experimental Method

Oral compositions (gel toothpastes) containing β-caryophyllene were prepared based on the formulae shown in Table 10 below.

TABLE 10

Oral composition formulae containing β-caryophyllene

| Ingredients | Example 8-1 | Example 8-2 | Example 8-3 |
|---|---|---|---|
| Polyoxyethylene (20) stearyl ether | 0.5% | 0.5% | 0.5% |
| Xylitol | 5% | 5% | 5% |
| Tocopherol acetate | 0.3% | 0.3% | 0.3% |
| Propylene glycol | 3% | 3% | 3% |
| 85% Glycerol | 10% | 10% | 10% |
| β-Caryophyllene | 1% | 5% | 10% |
| Xanthan gum | 1% | 1% | 1% |
| Sodium alginate | 0.9% | 0.9% | 0.9% |
| Water | 78.3% | 74.3% | 69.3% |

By using any of the above oral compositions, β-caryophyllene volatilized in the oral cavity was inhaled, and β-caryophyllene was delivered via the oral mucosa.

### Experiment 9: Cosmetic Products Containing β-Caryophyllene Sample Preparation

Dipropylene glycol, dioctyl carbonate, 1,2-pentanediol, glyceryl stearate, cetyl ethylhexanoate, polydimethylsiloxane, palmitoyl tripeptide-5, sucrose stearate, cetyl alcohol, octyldodecanol, dipeptide diaminobutyroyl benzylamide diacetate, myristyl myristate, acryloyldimethyltaurin ammonium/methacrylate beheneth-25 crosspolymer, phenoxyethanol, carbomer, arginine, and tocopherol were purchased from FUJIFILM Wako Pure Chemical Corporation.

### Experimental Method

Cosmetic products (face creams) containing β-caryophyllene were prepared according to the formulae shown in Table 11 below.

TABLE 11

Cosmetic product formulae containing β-caryophyllene

| Ingredients | Example 9-1 | Example 9-2 | Example 9-3 |
|---|---|---|---|
| β-Caryophyllene | 5.00% | 10.00% | 20.00% |
| Glycerol | 12.06% | 11.42% | 10.15% |
| Dipropylene glycol | 6.89% | 6.53% | 5.80% |
| Dioctyl carbonate | 8.61% | 8.16% | 7.25% |
| 1,2-Pentanediol | 4.31% | 4.08% | 3.63% |
| Glyceryl stearate | 5.17% | 4.89% | 4.35% |
| Cetyl ethylhexanoate | 5.17% | 4.89% | 4.35% |
| Polydimethylsiloxane | 5.17% | 4.89% | 4.35% |
| MCT | 5.17% | 4.89% | 4.35% |
| Palmitoyl tripeptide-5 | 3.96% | 3.75% | 3.34% |
| Sucrose stearate | 3.44% | 3.26% | 2.90% |
| Cetyl alcohol | 5.17% | 4.89% | 4.35% |
| Octyldodecanol | 2.07% | 1.96% | 1.74% |
| Dipeptide diaminobutyroyl benzylamide diacetate | 2.58% | 2.45% | 2.18% |
| Myristyl myristate | 18.94% | 17.95% | 15.95% |
| Acryloyldimethyltaurin ammonium/methacrylate beheneth-25 crosspolymer | 0.86% | 0.82% | 0.73% |
| Phenoxyethanol | 1.03% | 0.98% | 0.87% |
| Carbomer | 0.45% | 0.42% | 0.38% |
| Arginine | 0.34% | 0.33% | 0.29% |
| Artemia extract | 3.44% | 3.26% | 2.90% |
| Tocopherol | 0.17% | 0.16% | 0.15% |

By using any of the above cosmetic products, β-caryophyllene volatilized on the skin was inhaled, and β-caryophyllene was delivered (absorbed) through the skin.

### Experiment 10: Air Fresheners Containing β-Caryophyllene

### Experimental Method

A liquid air freshener containing 10 mL of β-caryophyllene was prepared and volatilized in a room.

The concentration of β-caryophyllene in the room in this case is presumed to be as follows.

### Example 10: Amount of β-Caryophyllene Volatilized from a β-Caryophyllene-Containing Air Freshener Placed in a Room

Consider the concentration of β-caryophyllene at equilibrium in air when β-caryophyllene is diffused in air. To this end, the value of the vapor pressure of β-caryophyllene at 25° C. needs to be determined. Assuming that the molar enthalpy of vaporization is not affected by temperature, the Clausius-Clapeyron equation expressing the relationship between vapor pressure (Pvap) and temperature (t) can be rewritten as:

$${p}_{v a p} = {p}_{0} exp \left[\frac{{Δ}_{v a p} {H}_{m}}{R} \left(\frac{1}{{T}_{0}} - \frac{1}{T}\right)\right] .$$

The value of vapor pressure P₀ at temperature T₀ needs to be a known value. Here, the boiling point of β-caryophyllene (the temperature at which the vapor pressure becomes equal to atmospheric pressure) is 130° C., and this can be said that, when T₀ is 403 K (130° C.), P₀ is 1.0 × 10⁵ Pa.

The molar enthalpy of vaporization (ΔvapHm) for β-caryophyllene is not reported, but considering that β-caryophyllene (boiling point: 130° C.) and octane (boiling point: 125.7° C.) are the same molecular species, that is, both hydrocarbon molecules and have similar boiling points, the molar enthalpy of vaporization for β-caryophyllene is presumed to be equivalent to the molar enthalpy of vaporization for octane, which is 35.0 kJ/mol. Substituting the values of T₀ and P₀ into the above equation gives a value of the vapor pressure of β-caryophyllene at 27° C., which is 2.5 × 10³ Pa. Therefore, when β-caryophyllene is abundantly present, the concentration of β-caryophyllene at equilibrium is 2.8 × 10³ Pa/1.0 × 10⁵ Pa = 2.8%.

Next, discuss the concentration of β-caryophyllene increasing toward equilibrium. The evaporation rate of β-caryophyllene is presumably proportional to the difference between the concentration of β-caryophyllene at equilibrium and the concentration of β-caryophyllene in air. Therefore, the evaporation rate of β-caryophyllene can be expressed as an exponential function that converges to the concentration of β-caryophyllene at equilibrium. The exponential function presumably represents a proportional relationship in the t = 0 limit as seen in Table 1. Considering that the 2.8% β-caryophyllene is equivalent to 3,232 µg/100 mL and that the slope in the t = 0 limit is 0.0739 [µg/100 mL/min] , the spatial concentration of volatilized β-caryophyllene in the case of hanging an absorbent cotton ball soaked with 10 mL of β-caryophyllene is expressed by:

$$\text{y} \text{=} \text{3232} \times \left(1 - {e}^{- \frac{t}{48985}}\right) .$$

The unit of y is µg/100 mL, and the unit of t is minutes. The half-life of this exponential function is 23.5 days, indicating that the volatilization rate of β-caryophyllene at room temperature is very slow.

In the case of using β-caryophyllene as an air freshener, the spatial concentration of volatilized β-caryophyllene is as follows. An example is given where 10 mL of β-caryophyllene is volatilized in a room. The volatilized β-caryophyllene is assumed to diffuse quickly throughout the room and reach a uniform concentration. The spatial concentration of β-caryophyllene in this case is also expressed by the same equation as above, that is,

$$\text{y} \text{=} \text{3232} \times \left(1 - {e}^{- \frac{t}{48985}}\right) .$$

The room is ventilated once every 360 minutes, and after ventilation, the concentration of β-caryophyllene is assumed to become zero. The length of the 360 minutes is short enough compared to the half-life of the exponential function, which is 23.5 days, so that the spatial concentration of β-caryophyllene over up to 360 minutes from start can be approximated as proportional to time, as expressed by:

$$y = 0.066 \times t .$$

Since a human inhales 500 mL of air every 4 seconds through breathing, the amount of β-caryophyllene inhaled by a human under 360-minute exposure to the spatial concentration of β-caryophyllene expressed by the above equation is:

$${\sum }_{t = 0}^{90} 0.066 \times 5 \times 4 t = 5390 \text{μ} \text{g} = 5.39 \text{mg} .$$

As shown in the results of Example 5-22 above, about 3 mg of β-caryophyllene was detected per cigarette in smoking β-caryophyllene capsule-containing cigarettes. On the other hand, the amount of β-caryophyllene inhaled per hour in the case of using air fresheners is smaller, but still seemingly sufficient.

### Experiment 11: Bioavailability of Inhaled β-Caryophyllene (in Capsules) in Smokers

As in Example 5-2 above, easily disintegrable seamless capsules containing 15% β-caryophyllene (liquid content: 19.3 mg) were prepared and individually entrapped in cigarette filters. The applicants sought to estimate the serum β-caryophyllene concentration in a subject smoking 20 cigarettes per day under the conditions that the subject breaks the capsule to allow its content to diffuse out prior to each smoking.

In the experiments using a mouse exposed to β-caryophyllene for 60 minutes, the amount of inhaled β-caryophyllene was 54 µg (3.0 mg/kg, Experiment 1A), whereas the serum concentration of β-caryophyllene was 102 ng/mL (102 ppb, Experiment 1B).

On the other hand, in the experiments using a smoking machine, the simulated amount of β-caryophyllene inhaled by a human is 0.29 mg per cigarette (0.41 µg/kg, Example 5-23). Generally, the serum concentration of β-caryophyllene is proportional to the amount of β-caryophyllene delivered per body weight. Therefore, the serum concentration of β-caryophyllene in a human after smoking one cigarette is presumably 0.14 ng/mL (0.14 ppb).

When the one cigarette is smoked per hour, assuming that the half-life of the serum concentration is 85.4 minutes, the serum concentration will be plotted in the graph as shown in FIG. 6.

Therefore, the average daily serum concentration of β-caryophyllene is 0.24 ng/mL (0.24 ppb).

### Experiment 12: Blood Pressure Reducing Effect of β-Caryophyllene Delivery

As mentioned above, the applicants confirmed that inhalation delivery of β-caryophyllene promotes relaxation. In addition, the applicants anticipated blood pressure reducing effect and actually monitored blood pressure. The results demonstrated blood pressure reducing effect of β-caryophyllene delivered by smoking after breakage of easily disintegrable capsules embedded in cigarette filters, vaping of e-cigarettes, oral ingestion of capsules, inhalation with air fresheners, and inhalation and dermal absorption with cosmetic products.

### INDUSTRIAL APPLICABILITY

The present invention provides a novel agent or composition and others.

1-4. (canceled)

5. A composition comprising caryophyllene and a flavoring agent.

6. (canceled)

7. The composition according to claim 5, wherein the amount of the caryophyllene is 1 mass% or more relative to the total mass of the composition which is assumed as 100 mass%.

8. A capsule comprising caryophyllene.

9. The capsule according to claim 8, further comprising a core and a shell, wherein the core comprises the caryophyllene.

10-12. (canceled)

13. The capsule according to claim 9, wherein the core comprises 1 mass% or more of the caryophyllene relative to the total mass of the core which is assumed as 100 mass%.

14-15. (canceled)

16. The composition according to claim 5, wherein the caryophyllene comprises a caryophyllene extracted or concentrated from clove, caraway, basil, oregano, hop, cinnamon, Ceylon cinnamon, rosemary, cannabis, hemp, Cannabis sativa, black pepper, lavender, malabathrum, ylang-ylang, copaiba, melegueta pepper, or another essential oil.

17. The composition according to claim 5, wherein the caryophyllene comprises a chemically synthesized caryophyllene.

18-25. (canceled)

26. A perfumery, cosmetic, food or drink product containing comprising caryophyllene.

27. The perfumery, cosmetic, food or drink product according to claim 26, wherein the product is an air freshener.

28. The perfumery, cosmetic, food or drink product according to claim 26, wherein the product is an oral care product.

29. The perfumery, cosmetic, food or drink product according to claim 26, wherein the product is a cosmetic product.

30. (canceled)

31. The perfumery, cosmetic, food or drink product according to claim 26, wherein the product is a food or drink product in the form of a capsule.

32. (canceled)

33. A method for promoting relaxation, promoting sleep, prolonging a resting-state time, prolonging a motionless time, and/or preventing blood pressure elevation,
the method comprising administering caryophyllene to a subject in need thereof.

34. The method according to claim 33, wherein the method is a method for promoting sleep comprising administering caryophyllene to the subject in need thereof.

35. The method according to claim 33, wherein the method is a method for preventing blood pressure elevation comprising administering caryophyllene to the subject in need thereof.

36. The method according to claim 33, wherein the administering is at least one selected from the group consisting of oral administration, pulmonary administration, and dermal administration.

37. The method according to claim 33, wherein the administering is pulmonary administration.

38. The method according to claim 33, wherein the administering is pulmonary administration using a capsule or a filter comprising caryophyllene.

39. The method according to claim 33, wherein the administering is pulmonary administration using at least one product or device comprising caryophyllene, which is selected from the group consisting of a tobacco product, an inhalation device, a perfumery product, and a cosmetic product.

40. The method according to claim 33, wherein the administering is pulmonary administration comprising breaking a capsule comprising a core and a shell, wherein the core comprises the caryophyllene.
